(19) United States
(12) Patent Application Publication
(10) Pub. No.: US 2009/0209001 A1
(43) Pub. Date: Aug. 20, 2009

(54) 2-Deoxy-D-Ribose 5-Phosphate Aldolases (DERAS) And Uses Thereof

(75) Inventors: Martin Schüermann, Jülich (DE); Marcel Gerhardus Wubbolts, Sittard (NL); Daniel Mink, Eupen (BE); Michael Wolberg, Jülich (DE); Johannes Helena Michael Mommers, Einighausen (NL); Stefan Martin Jennewein, Alsdorf (DE)

Correspondence Address: NIXON & VANDERHYE, PC, 901 NORTH GLEBE ROAD, 11TH FLOOR, ARLINGTON, VA, 22203, US

(73) Assignee: DSM IP ASSET B.V., Te Heerlen (NL)

(21) Appl. No.: 11/628,232

(22) PCT Filed: Jun. 2, 2005
(86) PCT No.: PCT/EP2005/005989

§ 371 Date: May 27, 2008

Related U.S. Application Data
(60) Provisional application No. 60/578,655, filed on Jun. 10, 2004.

(30) Foreign Application Priority Data
Jun. 4, 2004 (EP) 04076639.6

(51) Int. Cl. C12P 21/00; C12N 9/88; C40B 30/00; C07H 21/00; C12N 15/63; C12N 1/00; C12P 19/02

(52) U.S. Cl. 435/69.1; 435/232; 506/7; 536/23.1; 536/23.2; 435/320.1; 435/243; 435/105

(57) ABSTRACT

The invention relates to isolated mutants of enzymes from the group of 2-deoxy-D-ribose 5-phosphate aldolase wild-type enzymes having a productivity factor (as determined by a specific test) which is at least 10% higher than the productivity factor for the corresponding wild-type enzyme from which it is a mutant. The mutants have at least one amino acid substitution at one or more of the positions corresponding to K13, T19, Y49, N80, D84, A93, E127, A128, K146, K160, I166, A174, M185, K196, F200, and S239 in Escherichia coli K12 (EC 4.1.2.4) wild-type enzyme sequence, and/or a deletion of at least one amino acid at the positions corresponding to S258 and Y259 therein, optionally combined with, specific, C-terminal extension and/or N terminal extension.

The invention also relates to screening processes to find 2-deoxy-D-ribose 5-phosphate aldolase enzymes (either as such or as mutants) having a productivity factor (as determined by said specific test, which forms an essential part of the screening) which is at least 10% higher than the reference value.

Moreover, the invention relates to mutant enzymes obtained by the screening process, and to nucleic acids encoding such mutants, and to vectors and host cells comprising, respectively, such nucleic acids or mutants.

Finally the invention relates to the use of such (preferably mutant) enzymes, nucleic acids, vectors and host cells in the production of, for instance, 6-chloro-2,4,6-trideoxy-D-erythrohexapyranoside.

The invention relates to isolated mutants of enzymes from the group of 2-deoxy-D-ribose 5-phosphate aldolase wild-type enzymes from natural sources belonging to the group consisting of eukaryotic and prokaryotic species, each such wild-type enzyme having a specific productivity factor, as determined by the DERA Productivity Factor Test, in the production of 6-chloro-2,4,6-trideoxy-D-erythrohexapy-ranoside (hereinafter also referred to as CTeHP) from an at least equimolar mixture of acetaldehyde and chloroacetaldehyde. As meant herein, an improved productivity factor means the combined (and favorable) result of changes in resistance, catalytic activity and affinity of such aldolases towards an α-Leaving-Group substituted acetaldehyde and acetaldehyde. The method of determining the said productivity factor is described in the experimental part hereof, and will hereinafter be referred to as the “DERA Productivity Factor Test” (hereinafter sometimes also referred to as DPFT). Wild-type enzymes are enzymes as they can be isolated from natural sources or environmental samples; naturally occurring mutants of such enzymes (i.e. mutants as also can be isolated from natural sources or environmental samples, within the scope of this patent application are also considered to be wild-type enzymes. The term mutants, for this patent application, therefore solely will intend to indicate that they have been or are being obtained from wild-type enzymes by purposive mutations of the DNA (nucleic acid) encoding said wild-type enzymes (whether by random mutagenesis, for instance with the aid of PCR or by means of UV irradiation, or by site-directed mutation, e.g. by PCR methods, saturation mutagenesis etc. as are well-known to the skilled man, optionally with recombination of such mutations, for instance by a recombination technique as described in WO/010311).

In nature 2-deoxy-D-ribose 5-phosphate aldolases, e.g. the 2-deoxy-D-ribose 5-phosphate aldolase from E. coli K12 (DERA, EC 4.1.2.4), are known to enantioselectively catalyze the (reversible) aldol reaction between acetaldehyde and D-glyceraldehyde 3-phosphate to form 2-deoxy-D-ribose 5-phosphate. Any enzyme being capable of enantioselectively catalyzing this reaction, for the purposes of this patent application, or being capable of enantioselectively catalyzing the formation of a 2,4,6-trideoxyhexose from an α-Leaving-Group substituted acetaldehyde and acetaldehyde, is said to have DERA activity.

As described in—for instance—U.S. Pat. No. 5,795,749, the synthesis of certain 2,4,6-trideoxyhexoses can be accomplished by the use of a 2-deoxy-D-ribose 5-phosphate aldolase as an enantioselective catalyst. In said process use is made of acetaldehyde and a 2-substituted aldehyde as reactants, and the reaction proceeds via a 4-substituted 3-hydroxybutanal intermediate. Accordingly, 2-deoxy-D-ribose 5-phosphate aldolase, for instance, can be used—as described by Gijsen & Wong in JACS 116 (1994), page 8422—in a process for the synthesis of the hemiacetal 6-chloro-2,4,6-trideoxy-D-erythrohexapyranoside. This hemiacetal compound is herein, as mentioned before, also referred to as CTeHP. It is a suitable intermediate in the production of certain (4R,6S)-2-(6-substituted-1,3-dioxane-4-yl)acetic acid derivatives, for instance the t-butyl ester thereof, which in the present application will be referred to as CtBDAc. Such 2,4,6-trideoxyhexoses and 6-halo- or 6-cyano-substituted derivatives thereof, as well as such (4R,6S)-2-(6-substituted-1,3-dioxane-4-yl)acetic acid derivatives, and further compounds that can be considered to be equivalent thereto, are valuable chiral building blocks in the production of important groups of pharmaceutical products with cholesterol-lowering properties or anti-tumor properties. Important examples of such pharmaceuticals are the so-called statins like, for instance, the vastatins rosuvastatin (Crestor®; a trade name of Astra Zeneca) or atorvastatin (Lipitor®; a trade name of Pfizer). Other examples of statins are lovastatin, cerivastatin, simvastatin, pravastatin and fluvastatin. The statins generally are known to function as so-called HMG-CoA reductase inhibitors. Moreover, various derivatives of such pharmaceutical compounds (or intermediates thereof) are known to be interesting as well, for instance the hemiacetal 6-cyano-2,4,6-trideoxy-D-erythrohexapyranoside, which in the present application will be referred to as CyTeHP, which possibly is an alternative intermediate for the production of atorvastatin.

As mentioned in WO 03/006656, a known disadvantage of the enzyme catalyzed aldol condensations of U.S. Pat. No. 5,795,749 (cited above) is that the production capacity is low. It has thus successfully been attempted in WO 03/006656 to overcome such problems of low production capacity by performing the reaction at relatively high concentrations of reactants and by the preferred use of the 2-deoxy-D-ribose 5-phosphate aldolase from E. coli K12 (DERA, EC 4.1.2.4) in combination with α-chloroacetaldehyde as preferred substrate next to acetaldehyde.

Nevertheless, as the present inventors observed in their studies leading to the present invention, DERA enzymes so far, unfortunately, show rather poor resistance to aldehyde substrates (especially towards acetaldehyde and—even more pronounced—towards α-L-substituted acetaldehyde). In particular, if the leaving group L is chloro very high deactivation of the DERA enzymes is observed at concentrations useful for the biosynthesis of trideoxyhexoses. Moreover, as the inventors found, the known 2-deoxy-D-ribose 5-phosphate aldolase enzymes appear to have very low affinity and activity towards the substrate chloroacetaldehyde. For those reasons, in fact, relatively high amounts of (expensive) DERA enzymes are required to obtain good synthesis reaction yields. Accordingly, there was substantial need for finding DERA enzymes having an improved productivity factor (i.e. the combined result of changes in resistance, catalytic activity of such aldolases towards α-L-substituted acetaldehyde and acetaldehyde should be favourable). And of course, preferably also the production capacity of synthesis routes to trideoxyhexoses should be improved.

It is to be noticed that a recent article from W. A. Greenberg et al., in PNAS, vol. 101, p. 5788-5793 (2004) describes attempts to find wild type DERA enzymes with improved volumetric productivity in the DERA reaction and disclose the amino acid sequence of a wild type DERA from an unknown source organism. As will be discussed hereinafter, the article also describes specific ways for the screening methods to find DERA enzymes. However, the authors focus on substrate inhibition and do not really address the problems inherent to the use of DERA enzymes in combination with (relatively high) concentrations of, for instance, chloroacetaldehyde, namely strong deactivation of the enzymes. In fact, the authors try to minimize substrate inhibition problems by feeding the substrates at the same rate as they are being taken away by the reaction.

As mentioned above, in nature 2-deoxy-D-ribose 5-phosphate aldolase enantioselectively catalyzes the (reversible) aldol reaction between acetaldehyde and D-glyceraldehyde 3-phosphate to form 2-deoxy-D-ribose 5-phosphate. For the purposes of the present patent application this natural reaction, and more precisely the reverse reaction thereof (i.e. the degradation of 2-deoxy-D-ribose 5-phosphate into acetaldehyde and D-glyceraldehyde 3-phosphate) will be used as one of the reference reactions for establishing resistance, c.q. stability, data for the mutant enzymes provided. This degradation reaction therefore hereinafter will be referred to as the DERA natural substrate reaction. However, in addition to the DERA natural substrate reaction, for assessment of productivity of the mutant enzymes also a further test assay reaction, namely the DERA Productivity Factor Test (DPFT), with chloroacetaldehyde and acetaldehyde as substrates, will be used. As indicated before, productivity represents the combined (i.e. net) effects of changes in activity, resistance (stability) and affinity.

In the context of the present invention, the resistance and productivity of the DERA mutants at each occurrence in particular will be compared with that of the wild-type enzyme from which the mutant is derived, and/or will be compared with that of the E. coli K12 DERA (a wild-type DERA), in said DERA natural substrate reaction and/or DPFT reaction.

Preferably, in the comparison of the specific productivity factors of two enzymes, identical conditions are used. With ‘identical conditions’ is meant that except for the different nucleic acid sequences encoding the two different enzymes, there are substantially no differences in set-up between the two DERA Productivity Factor Tests. This means that parameters, such as for instance temperature, pH, concentration of cell-free extract (cfe), chloracetaldehyde and acetaldehyde; genetic background such as an expression system, i.e expression vector and host cell etc are preferably all kept identical.

As meant herein, the term improved productivity factor is thus the (favorable) resultant of changes in resistance, catalytic activity and affinity, under standard testing conditions as described in the experimental part hereof, especially taking into consideration the results of the DPFT reaction. The productivity factor as used in the present application, therefore more precisely corresponds to the CTeHP formation value. The DERA mutants provided according to the present invention are at least 10% more productive than the wild-type DERA enzyme from which it is a mutant, and/or than the E. coli K12 DERA, in the DERA natural substrate reaction and/or DPFT reaction. Accordingly, they have a substantially better resistance (i.e. they remain at a higher percentage of their activity level for a given period of time) in the presence of an α-Leaving-Group substituted acetaldehyde and acetaldehyde, or usually are substantially more active in the natural substrate DERA reaction.

The present invention further in particular relates to a process for the screening for wild-type enzymes from the group of 2-deoxy-D-ribose 5-phosphate aldolase enzymes having a productivity factor, as determined by the DERA Productivity Factor Test, in the production of 6-chloro-2,4,6-trideoxy-D-erythrohexapyranoside (CTeHP) from an at least equimolar mixture of acetaldehyde and chloroacetaldehyde, which is at least 10% higher than the productivity factor for the 2-deoxy-D-ribose 5-phosphate aldolase enzyme from Escherichia Coli K12 (EC 4.1.2.4) having a wild-type enzyme sequence of [SEQ ID No. 1].

The present invention further in particular also relates to a process for the screening for mutant enzymes from the group of 2-deoxy-D-ribose 5-phosphate aldolase enzymes having a productivity factor, as determined by the DERA Productivity Factor Test, in the production of 6-chloro-2,4,6-trideoxy-D-erythrohexapyranoside (CTeHP) from an at least equimolar mixture of acetaldehyde and chloroacetaldehyde, which is at least 10% higher than the productivity factor for the corresponding wild-type enzyme. More particularly it also relates to a process for the screening for enzymes from the group of 2-deoxy-D-ribose 5-phosphate aldolase enzymes having such a productivity factor, that is at least 10% higher than the productivity factor for the 2-deoxy-D-ribose 5-phosphate aldolase enzyme from Escherichia coli K12 (EC 4.1.2.4) having a wild-type enzyme sequence of [SEQ ID No. 1]. This sequence of [SEQ ID No. 1] is shown hereinafter in the sequence listings under the entry <400> 1.

As meant herein, the term mutant (enzyme) is intended to encompass such mutants as are obtained by genetic engineering of the DNA (nucleic acid) encoding a wild-type DERA enzyme and resulting for instance in replacements or substitutions, deletions, truncations and/or insertions in the amino acid sequence, for instance in the nucleic acid of [SEQ ID No. 6] (see sequence listing, under the entry <400> 6) encoding wild-type DERA enzyme from E. Coli K12) of a wild-type DERA enzyme, for instance the E. coli K12 DERA.

The present invention still further relates to isolated nucleic acids encoding such 2-deoxy-D-ribose 5-phosphate mutant aldolases having a higher and improved productivity factor when compared with the wild-type DERA enzyme from which it is a mutant, and/or compared with the E. coli K12 DERA; and to vectors comprising such isolated nucleic acids encoding the 2-deoxy-D-ribose 5-phosphate mutant aldolases according to the invention; and to host cells comprising such nucleic acids and/or vectors.

Finally, the present invention also relates to improved synthesis of pharmaceutical products as mentioned hereinabove, and of their derivatives and intermediates, by using 2-deoxy-D-ribose 5-phosphate mutant aldolases according to the invention, or by using nucleic acids encoding such mutants, or by using vectors comprising such nucleic acids, or by using host cells comprising such nucleic acids and/or vectors.

The present inventors, after detailed studies, have found that a vast amount of mutant DERA enzymes having an improved productivity factor when used in production of 6-chloro-2,4,6-trideoxy-D-erythrohexapyranoside (CTeHP) has become accessible. Namely the inventors have found that isolated mutants of enzymes from the group of 2-deoxy-D-ribose 5-phosphate aldolase wild-type enzymes can be obtained from natural sources belonging to the group consisting of eukaryotic and prokaryotic species, said wild-type enzymes each having a specific productivity factor, as determined by the DERA Productivity Factor Test, in the production of CTeHP from an at least equimolar mixture of acetaldehyde and chloroacetaldehyde, wherein the isolated mutants have a productivity factor which is at least 10% higher than the productivity factor for the corresponding wild-type enzyme from which it is a mutant and wherein the productivity factors of both the mutant and the corresponding wild-type enzyme are measured under identical conditions.

The isolated mutants of enzymes from the group of 2-deoxy-D-ribose 5-phosphate aldolase wild-type enzymes (DERAs) according to the invention can be either derived from DERAs from eukaryotic origin or, as is more preferred, from prokaryotic origin. When the DERAs are from eukaryotic origin, they are obtained from organisms consisting of one or more eukaryotic cells that contain membrane-bound nuclei as well as organelles. Eukaryotic cells, for instance, can be cells from humans, animals (e.g. mice), plants and fungi and from various other groups, which other groups collectively are referred to as “Protista”. Suitable DERAs, for instance, can be obtained from eukaryotic sources belonging to the Metazoa, i.e. from animals except sponges and protozoans, for instance from nematodes, arthropodes and vertebrates, e.g. from Caenorhabditis elegans, Drosophila melanogaster, Mus musculus, and Homo sapiens.

More preferably, however, the isolated mutant DERAs according to the present invention are from prokaryotic origin, i.e. from single-cell organisms without a nucleus generally belonging to the kingdoms of Archaea (comprising the phyla Crenarchaeota and Euryarchaeota) and Bacteria.

A survey of the phylogenetic tree for species belonging to the kingdom of Archaea, from which species suitable DERA mutants according to the invention can be obtained, is presented in table 1. Most preferably, the isolated mutant DERAs according to the present invention are from bacterial origin. A survey of the phylogenetic tree for species belonging to the kingdom of Bacteria, from which species suitable DERA mutants according to the invention can be obtained, is presented in table 2. In Table 1 and 2 GI stands for generic identifier for the retrieval of amino acid sequences from the NCBI Entrez browser; the number after GI: can be used to access the amino acid sequences of the wild-type DERAs and nucleic acid sequences encoding said amino acid sequences, for instance by using the numbers in a database accessible via the following site/search engine: NCBI (http://www.ncbi.nlm.nih.gov).

The person skilled in the art is aware that wild-type DERA amino acid sequences and nucleic acid sequences encoding these wild-type DERAs other than those mentioned in table 1 and 2 can easily be found in a manner known per se in protein and nucleic acid databases, for example using the site/search engine mentioned above.

Within the kingdom of Bacteria the mutant DERAs most preferably are based on wild type DERAs originating from the phylum Proteobacteria, and therein more specifically from the class of Gamma-proteobacteria, especially from the order of Enterobacteriales to which also the family of Enterobacteriaceae belongs. Said family inter alia includes the genus Escherichia.

Accordingly, suitable mutant DERAs for use in the context of the present invention, for instance, can be obtained by purposive mutations of the DNA encoding said wild type enzymes from the prokaryotic sources as are being summarized in table 3, in—roughly—an increasing (from about 20% identity to 100% identity) identity percentage with Escherichia coli K12.

TABLE 1
Archaea

| | | | | | | Generic |
|---|---|---|---|---|---|---|
| | | | | | | identifier |
| Phylum | Class | Order | Family | Genus | species | (GI) |
| Euryarchaeota | Thermoplasmata | Thermoplasmatales | Thermoplasmataceae | Thermoplasma | volcanium | 24636808 |
| | | | | Thermoplasma | acidophilum | 13878466 |
| | Thermococci | Thermococcales | Thermococcaceae | Thermococcus | kodakaraensis | 34395642 |
| | Methanobacteria | Methanobacteriales | Methanobacteriaceae | Methanothermobacter | thermoautotrophicus | 3913443 |
| | Halobacteria | Halobacteriales | Halobacteriaceae | Halobacterium | sp. NRC-1 | 24636814 |
| Crenarchaeota | Thermoprotei | Desulfurococcales | Desulfurococcaceae | Aeropyrum | pernix | 24638457 |
| | | Thermoproteales | Thermoproteaceae | Pyrobaculum | aerophilum | 24636804 |

TABLE 2
Bacteria

| | | | | | | | Generic |
|---|---|---|---|---|---|---|---|
| | | | | | | | identifier |
| Phylum | Class | Order | Family | Genus | species | strain | (GI) |
| Aquificae | Aquificae | Aquificales | Aquificaceae | Aquifex | aeolicus | VF5 | 3913447 |
| Thermotogae | Thermotogae | Thermotogales | Thermotogaceae | Thermotoga | maritima | MSB8 | 7674000 |
| Spirochaetes | Spirochaetes | Spirochaetales | Spirochaetaceae | Treponema | pallidum | Nichols | 7673994 |
| Deinococcus- | Deinococci | Deinococcales | Deinococcaceae | Deinococcus | radiodurans | R1 | 24636816 |
| Thermus | | | | | | | |
| Cyanobacteria | | Chroococcales | | Synechocystis | sp. PCC 6803 | | 3913448 |
| | | Nostocales | Nostocaceae | Nostoc | sp. PCC 7120 | | 24636799 |
| Actinobacteria | Actinobacteria | Actinomycetales | Streptomycetaceae | Streptomyces | coelicolor | A3(2) | 13162102 |
| | | | Corynebacteriaceae | Corynebacterium | glutamicum | ATCC 13032 | 24636791 |
| | | | Mycobacteriaceae | Mycobacterium | tuberculosis | H37Rv | 1706364 |
| | | | | Mycobacterium | leprae | TN | 13878464 |
| Firmicutes | Bacilli | Bacillales | Bacillaceae | Bacillus | subtilis | 168 | 1706363 |
| | | | | Bacillus | halodurans | JCM 9153 | 13878470 |
| | | | | Bacillus | cereus | ATCC 14579 | 38372184 |
| | | | | Bacillus | anthracis | Ames | 38372187 |
| | | | | Listeria | innocua | CLIP 11262 | 22095578 |
| | | | | Listeria | monocytogenes | EGD-e | 22095575 |
| | | | | Oceanobacillus | iheyensis | HTE831 | e.g. 38372231 |
| | | | Staphylococcaceae | Staphylococcus | aureus | MW2 | e.g. 24636793 |
| | | | | Staphylococcus | epidermidis | ATCC 12228 | 38257566 |
| | | Lactobacillales | Lactobacillaceae | Lactobacillus | plantarum | WCFS1 | 38257534 |
| | | | Streptococcaceae | Streptococcus | pyogenes | SF370 | 24636813 |
| | | | | Streptococcus | pneumoniae | ATCC | 22095579 |
| | | | | | | BAA-334 | |
| | | | | Lactococcus | Lactis; subsp. lactis | IL1403 | 13878465 |
| | | | Enterococcaceae | Enterococcus | faecalis | V583 | 46576519 |
| | Clostridia | Clostridiales | Clostridiaceae | Clostridium | perfringens | 13 | 22095574 |
| | | | | Clostridium | acetobutylicum | VKM B-1787 | 24636809 |
| | | Thermoanaero- | Thermoanaero- | Thermoanaero- | tengcongensis | MB4 | 22095572 |
| | | bacteriales | bacteriaceae | bacter | | | |
| | Mollicutes | Mycoplasmatales | Mycoplasmataceae | Mycoplasma | pneumoniae | M129 | 118445 |
| | | | | | | UAB CTIP | |
| | | | | Mycoplasma | pulmonis | | 24636810 |
| | | | | Mycoplasma | pirum | BER | 1352232 |
| | | | | Mycoplasma | genitalium | G-37 | 1352231 |
| | | | | Mycoplasma | hominis | FBG | 1169269 |
| | | | | Ureaplasma | parvum | Serovar 3 | 13878474 |
| Proteobacteria | Alphaproteo- | Rhizobiales | Rhizobiaceae | Agrobacterium | tumefaciens | C58 | 24636797 |
| | bacteria | | | Sinorhizobium | meliloti | 1021 | 24636806 |
| | Betaproteo- | Burkholderiales | Burkholderiaceae | Burkholderia | mallei | ATCC 23344 | |
| | bacteria | | | Burkholderia | pseudomallei | ATCC 23343 | |
| | | Neisseriales | Neisseriaceae | Chromobacterium | violaceum | DSM 30191 | 39930965 |
| | Gammaproteo- | Pseudomonadales | Pseudomonaceae | Pseudomonas | syringae | DC3000 | 28851430 |
| | bacteria | Alteromonadales | Alteromonadaceae | Shewanella | oneidensis | MR-1 | 39931142 |
| | | Pasteurellales | Pasteurellaceae | Pasteurella | multicoda | Pm70 | 13431461 |
| | | | | Haemophilus | influenzae | Rd | 1169268 |
| | | | | Haemophilus | ducreyi | 35000HP | 39931016 |
| | | Vibrionales | Vibrionaceae | Vibrio | cholerae | El Tor | 13878471 |
| | | | | | | N16961 | |
| | | | | Vibrio | vulnificus | CMCP6 | 39931134 |
| | | | | Vibrio | parahaemolyticus | RIMD | 39931108 |
| | | | | | | 2210633 | |
| | | Enterobacteriales | Enterobacteriaceae | Yersinia | pestis | CO-92 | e.g. 24636801 |
| | | | | Photorhabdus | luminescens | TT01 | 39930948 |
| | | | | Shigella | flexneri | 2457T | 39931101 |
| | | | | Salmonalla | typhi | Ty2 | 24636800 |
| | | | | Salmonalla | typhimurium | LT2 | 24636803 |
| | | | | Escherichia | coli | K12 | 729314 |
| | | | | Escherichia | coli | CFT073 | 26251271 |
| | | | | Escherichia | coli | 0157: H7 | 24636798 |

Table 3: Prokaryotic sources for suitable mutant DERAs:

Thermoplasma volcanium, Thermoplasma acidophilum, Aeropyrum pernix, Aquifex aeolicus, Sinorhizobium meliloti, Oceanobacillus iheyensis, Pyrobaculum aerophilum, Thermococcus kodakaraensis, Lactobacillus plantarum, Methanothermobacter thermoautotrophicus, Mycoplasma pneumoniae, Mycoplasma pirum, Mycoplasma genitalium, Mycoplasma hominis, Mycoplasma pulmonis, Thermotoga maritima, Synechocystis sp. PCC 6803, Treponema pallidum, Streptococcus pyogenes, Streptococcus pneumoniae, Nostoc sp. PCC 7120, Halobacterium sp. NRC-1, Haemophilus influenzae, Haemophilus ducreyi, Yersinia pestis, Ureaplasma parvum, Staphylococcus aureus subsp. aureus Mu50, respectively subsp. aureus MW2, Staphylococcus epidermidis, Pasteurella multicoda, Mycobacterium tuberculosis, Mycobacterium leprae, Lactococcus lactis subsp. lactis, Enterococcus faecalis, Corynebacterium glutamicum, Thermoanaerobacter tengcongensis, Bacillus subtilis, Bacillus halodurans, Bacillus cereus, Bacillus anthracis strain Ames, Listeria innocua, Listeria monocytogenes, Clostridium perfringens, Clostridium acetobutylicum, environmental samples as mentioned in the article of W. A. Greenberg et al. in PNAS, vol. 101, p. 5788-5793 (2004), Deinococcus radiodurans, Pseudomonas syringae, Streptomyces coelicolor, Agrobacterium tumefaciens strain C58, Burkholderia mallei, Burkholderia pseudomallei, Chromobacterium violaceum, Shewanella oneidensis, Vibrio cholerae, Vibrio vulnificus, Vibrio parahaemolyticus, Photorhabdus luminescens, Salmonella typhi, Salmonella typhimurium, Shigella flexneri, Escherichia coli O157:H7, Escherichia coli CFT073, Escherichia coli K12.

A very suitable wild-type reference DERA for comparing the specific productivity factor of the mutant DERAs as are obtained according to the present invention, is the 2-deoxy-D-ribose 5-phosphate aldolase from Escherichia coli K12 (EC 4.1.2.4) having, from N-terminus to C-terminus, a wild-type enzyme sequence of [SEQ ID No. 1]:

```
                10 20 30 40 50 60
MTDLKASSLR ALKLMDLNTL NDDDTDEKVI ALCHQAKTPV GNTAAICIYP RFIPIARKTL
                70 80 90 100 110 120
KEQGTPEIRI ATVTNFPHGN DDIDIALAET RAAIAYGADE VDVVFPYRAL MAGNEQVGFD
              130 140 150 160 170 180
LVKACKEACA AANVLLKVII ETGELKDEAL IRKASEISIK AGADFIKTST GKVAVNATPE
              190 200 210 220 230 240
SARIMMEVIR DMGVEKTVGF KPAGGVRTAE DAQKYLAIAD ELFGADWADA RHYRFGASSL
              250 259
LASLLKALGH GDGKSASSY
```

Therefore, the invention further relates to isolated mutants of enzymes from the group of 2-deoxy-D-ribose 5-phosphate aldolase wild-type enzymes from natural sources belonging to the group consisting of eukaryotic and prokaryotic species, each such wild-type enzyme having a specific productivity factor, as determined by the DERA Productivity Factor Test, in the production of chloro-2,4,6-trideoxy-D-erythrohexapyranoside (CTeHP) from an at least equimolar mixture of acetaldehyde and chloroacetaldehyde, wherein the isolated mutants have a productivity factor which is at least 10% higher than the productivity factor for the corresponding wild-type enzyme from which it is a mutant and wherein the productivity factors of both the mutant and the corresponding wild-type enzyme are measured under identical conditions and wherein the isolated mutants have a productivity factor which is at least 10% higher than the productivity factor for the 2-deoxy-D-ribose 5-phosphate aldolase from Escherichia coli K12 (EC4.1.2.4) having the wild type enzyme sequence of [SEQ ID No. 1] and wherein the productivity factors of both the mutant and the Escherichia coli K12 enzyme are measured under identical conditions.

It is to be noticed, that the wild-type sequence of the E. coli K12 (W3110) DERA enzyme (259 amino acids; [SEQ ID No. 1]), as well as the nucleotide sequence encoding said DERA enzyme (780 nucleotides, [SEQ ID No. 6]; see sequence listing), has been described by P. Valentin-Hansen et al. in “Nucleotide sequence of the deoC gene and the amino acid sequence of the enzyme”, Eur. J. Biochem. 125 (3), 561-566 (1982).

DeSantis et al., 2003, Bioorganic & Medicinal Chemistry 11, pp 43-52 disclose the design of five site-specific mutations of 2-deoxy-D-ribose 5-phosphate aldolase from E. coli (EC 4.1.2.4) in the phosphate binding pocket of the E. coli DERA: K172E, R207E, G205E, S238D and S239E. Of these mutant DERA enzymes, S238D and S239E are shown to have a higher activity towards its non-phosphorylated natural substrate (2-deoxy-D-ribose) than the wild type enzyme. These same mutants of E. coli 2-deoxy-D-ribose 5-phosphate aldolase are also disclosed in US 2003/0232416.

The present inventors have found, in sequence alignment studies using ClustalW, version 1.82 http://www.ebi.ac.uk/clustalw multiple sequence alignment at default settings (matrix: Gonnet 250; GAP OPEN: 10; END GAPS: 10; GAP EXTENSION: 0.05; GAP DISTANCES: 8), that the DERAs from eukaryotic and prokaryotic origin as can be used for deriving the isolated mutants according to the invention may vary over a broad range of identity percentage with the wild-type enzyme sequence of [SEQ ID No. 1] of the 2-deoxy-D-ribose 5-phosphate aldolase from Escherichia coli K12 (EC 4.1.2.4). Even at an identity percentage of about 20% still very suitable DERAs are being found that can be used as starting point for obtaining the mutants according to the present invention.

The inventors have found, that all DERAs as can be used in the present invention (and the mutants derived therefrom) all have in common, that they have at least eight conserved amino acids, namely F76, G79, E100, D102, K167, T170, K201, and G204, when being compared to the wild-type enzyme sequence of [SEQ ID No. 1]. Accordingly, all mutations as described below are at positions different from these conserved positions. It may be noticed, that K167 is the essential active site lysine which forms the Schiff base intermediate with acetaldehyde; K201 and D102 are involved in the catalytic proton relay system “activating” K167 according to Heine et al. in “Observation of covalent intermediates in an enzyme mechanism at atomic resolution”, Science 294, 369-374 (2001). The other five residues have not been described to be conserved or important for e.g. substrate recognition or catalysis, up to now.

Preferably, the isolated mutant DERAs have a productivity factor which is at least 10% higher than the productivity factor for the corresponding wild-type enzyme from which it is a mutant. The productivity factor is preferably at least 20%, more preferably at least 30%, still more preferably at least 40%, with even more preference at least 50%, more preferably at least 100%, even more preferably at least 200%, even more preferably at least 500%, even more preferably at least 1000%, even more preferably at least 1500% higher than for the corresponding wild-type enzyme.

More preferably, the isolated mutant DERAs have a productivity factor which is at least 10% higher than the productivity factor for E. coli K12 DERA. The productivity factor is preferably at least 20%, more preferably at least 30%, still more preferably at least 40%, with even more preference at least 50%, more preferably at least 100%, even more preferably at least 200%, even more preferably at least 500%, even more preferably at least 1000%, even more preferably at least 1500% higher than for E. coli K12 DERA.

A very important group of isolated mutants, that has been shown to be very effective in the intended reaction, are the isolated mutants of the 2-deoxy-D-ribose 5-phosphate aldolase from Escherichia coli K 2 (EC 4.1.2.4) having a wild-type enzyme sequence of [SEQ ID No. 1]. These isolated mutant DERAs have a productivity factor which is at least 10% higher than the productivity factor for the enzyme sequence of [SEQ ID No. 1]. The productivity factor is preferably at least 20%, more preferably at least 30%, still more preferably at least 40%, with even more preference at least 50%, and even more preferably at least 100%, even more preferably at least 200%, even more preferably at least 500%, even more preferably at least 1000%, even more preferably at least 1500% higher than that for enzyme sequence of [SEQ ID No. 1].

The present inventors have found that very suitable isolated mutant DERAs are being obtained when the mutants have at least one amino acid substitution at one or more of the positions K13, T19, Y49, N80, D84, A93, E127, A128, K146, K160, I166, A174, M185, K196, F200, or S239 in [SEQ ID No. 1], or at positions corresponding thereto, preferably at position F200 or at a position corresponding thereto, and/or a deletion of at least one amino acid at one of the positions S258 or Y259 in [SEQ ID No. 1], optionally in combination with C-terminal extension, preferably by one of the fragments TTKTQLSCTKW [SEQ ID No. 2] and KTQLSCTKW [SEQ ID No. 3] and/or in combination with N-terminal extension.

An example of a nucleic acid sequence encoding [SEQ ID No. 2] is given in [SEQ ID No. 7]. An example of a nucleic acid sequence encoding [SEQ ID No. 3] is given in [SEQ ID No. 8].

In one embodiment of the invention, site-directed mutations may be made by saturation mutagenesis performed on one of there above-mentioned positions in or corresponding to [SEQ ID No. 1], for instance on (the) position (corresponding to position) F200. With saturation mutagenesis is meant that the amino acid is substituted with every possible proteinogenic amino acid, for instance with alanine, arginine, aspartic acid, asparagine, cysteine, glutamic acid, glutamine, glycine, histidine, isoleucine, leucine, lysine, methionine, phenylalanine, proline, serine, threonine, tryptophan, tyrosine or valine, for instance by generating a library of variant enzymes, in which each variant contains a specific amino acid exchange at position 200 of [SEQ ID No. 1]. Preferably saturation mutagenesis is performed by exchanging the nucleic acid triplet encoding the amino acid to be substituted by every possible nucleic acid triplet, for example as described in example 4. Accordingly, these mutants have a sequence differing from that of [SEQ ID No. 1] (or of any other wild-type enzyme amino acid sequence from another natural source corresponding therewith at the identity percentage as found according to the above described ClustalW program) at one or more of the positions indicated, whilst still having the at least eight conserved amino acids, namely F76, G79, E100, D102, K167, T170, K201, and G204, discussed above. Thus, as meant herein, “corresponding mutations” are intended to indicate that these mutations occur in a specific “corresponding wild-type enzyme amino acid sequence” (i.e. a sequence of an enzyme having DERA activity).

Amino acid residues of wild-type or mutated protein sequences corresponding to positions of the amino acid residues in the wild-type amino sequence of the E. coli K12 DERA [SEQ ID No. 1] can be identified by performing ClustalW version 1.82 multiple sequence alignments (http://www.ebi.ac.uk/clustalw) at default settings (matrix: Gonnet 250; GAP OPEN: 10; END GAPS: 10; GAP EXTENSION: 0.05; GAP DISTANCES: 8). Amino acid residues which are placed in the same row as an amino acid residue of the E. coli K12 wild-type DERA sequence as given in [SEQ ID No. 1] in such alignments are defined to be positions corresponding to this respective amino acid residue of the E. coli K12 wild-type DERA [SEQ ID No. 1].

As used herein, the amino acids in the sequences and at the various positions therein, are indicated by their one letter code (respectively by their three letter code) as follows:

| One letter code | Three letter code | Name |
|---|---|---|
| A | ALA | Alanine |
| R | ARG | Arginine |
| D | ASP | Aspartic acid |
| N | ASN | Asparagine |
| C | CYS | Cysteine |
| E | GLU | Glutamic acid |
| Q | GLN | Glutamine |
| G | GLY | Glycine |
| H | HIS | Histidine |
| I | ILE | Isoleucine |
| L | LEU | Leucine |
| K | LYS | Lysine |
| M | MET | Methionine |
| F | PHE | Phenylalanine |
| P | PRO | Proline |
| S | SER | Serine |
| T | THR | Threonine |
| W | TRP | Tryptophan |
| Y | TYR | Tyrosine |
| V | VAL | Valine |

The above listed amino acids can be differentiated according to various properties, as may be important at specific positions in the sequence. Some of the amino acids, for instance, belong to the category of positively charged amino acids, namely especially lysine, arginine and histidine. Another category of amino acids is that of the hydrophilic amino acids, consisting of serine, threonine, cysteine, glutamine, and asparagine. Hydrophobic amino acids are isoleucine, leucine, methionine, valine, phenylalanine, and tyrosine. There is also a category of aromatic amino acids, namely phenylalanine, tyrosine and tryptophan. Still another possibility of categorizing the amino acids is according to their size: in order of decreasing size the amino acids can be listed as W>Y>F>R>K>L, I>H>Q>V>E>T>N>P>D>C>S>A>G.

Thus, each of the mutants claimed, is to be compared with the wild-type sequence from which it is derived. This means that a mutant according to the invention only can be considered to be a mutant when at least the first two of the following criteria are met:
(a) the mutation should be corresponding to one of the mutations indicated for E. coli K12;
(b) the mutation is not present in the wild-type enzyme from which the mutant is derived;
(c) at least eight conserved amino acids, namely F76, G79, E100, D102, K167, T170, K201, and G204, are still present at the corresponding positions.

Most preferably, the isolated mutant DERAs according to the present invention have at least one of the amino acid substitutions in, or corresponding to the substitutions in, [SEQ ID No. 1] selected from the group consisting of:
a. K13 and/or K196 replaced by a positively charged amino acid, preferably by R or H;
b. T19 and/or M185 replaced by another amino acid, preferably by another amino acid selected from the groups consisting of hydrophilic amino acids, in particular consisting of S, T, C, Q, and N, and/or hydrophobic amino acids, in particular consisting of V, L and I;
c. Y49 replaced by an aromatic amino acid selected from the group consisting of F and W;
d. N80 and/or 1166 and/or S239 replaced by another amino acid selected from the group of hydrophilic amino acids consisting of T, S, C, Q and N;
e. D84 and/or A93 and/or E127 replaced by another, preferably smaller, amino acid selected from the group of small amino acids consisting of, in order of decreasing size, E, T, N, P, D, C, S, A, and G;
f. A128 and/or K146 and/or K160 and/or A174 and/or F200 replaced by another amino acid selected from the group of hydrophobic amino acids consisting of I, L, M, V, F, and Y;
   and/or have a deletion of at least one amino acid at the positions S258 and Y259 in [SEQ ID No. 1], or at positions corresponding thereto,
   optionally in combination with C-terminal extension, preferably by one of the fragments TTKTQLSCTKW [SEQ ID No. 2] and KTQLSCTKW [SEQ ID No. 3] and/or in combination with N-terminal extension.

In one embodiment of the invention, in the isolated mutants of the invention the C-terminus may be truncated by deletion of at least one amino acid residue, e.g. by deletion of S258 and/or Y259 or of positions corresponding thereto and then extended, preferably by one of the fragments TTKTQLSCTKW [SEQ ID No. 2] and KTQLSCTKW [SEQ ID No. 3].

For clarity sake, the part “amino acid substitutions in, or corresponding to the substitutions in, [SEQ ID No. 1]” means that those substitutions either are substitutions in [SEQ ID No. 1], or are substitutions in a wild-type sequence other than that of E. coli K12 at positions corresponding to the ones that in E. coli would have been at the numbered positions.

Most preferably, the isolated mutant DERA has one or more of the mutations in, or corresponding to the mutations in, [SEQ ID No. 1] selected from the group of K13R, T19S, Y49F, N80S, D84G, A93G, E127G, A128V, K146V, K160M, I166T, A174V, M185T, M185V, K196R, F2001, F200M, F200V, S239C, ΔS258, ΔY259, C-terminal extension by TTKTQLSCTKW [SEQ ID No. 2], and C-terminal extension by KTQLSCTKW [SEQ ID No. 3].

As indicated here, the one letter code preceding the amino acid position number in [SEQ ID No. 1] indicates the amino acid as present in the said wild-type E. coli enzyme, and the one letter code following to the amino acid position number in [SEQ ID No. 1] indicates the amino acid as present in the mutant. The amino acid position number reflects the position number in the DERA of [SEQ ID No. 1] and any position corresponding thereto in other DERA wild types from other sources.

More in particular, the isolated mutant DERA has at least the following two mutations in, or corresponding to the two mutations in, [SEQ ID No. 1] selected from the group of F2001 and ΔY259; F200M and ΔY259; F200V and ΔY259; F200I and C-terminal extension by KTQLSCTKW [SEQ ID No. 3]; F200M and C-terminal extension by KTQLSCTKW [SEQ ID No. 3]; and F200V and C-terminal extension by KTQLSCTKW [SEQ ID No. 3];

The invention also relates to a process for the screening for wild-type enzymes from the group of 2-deoxy-D-ribose 5-phosphate aldolase enzymes having a productivity factor, as determined by the DERA Productivity Factor Test, in the production of 6-chloro-2,4,6-trideoxy-D-erythrohexapyranoside (CTeHP) from an at least equimolar mixture of acetaldehyde and chloroacetaldehyde, which is at least 10% higher than the productivity factor for the 2-deoxy-D-ribose 5-phosphate aldolase enzyme from Escherichia coli K12 (EC 4.1.2.4) having a wild-type enzyme sequence of [SEQ ID No. 1], wherein
(A) subsequently (i) total and/or genomic DNA and/or cDNA is isolated; (ii) an expression library of said isolated DNA is prepared, consisting of individual clones comprising said isolated DNA; (iii) the individual clones from the obtained expression library are incubated with a mixture of the substrates acetaldehyde and chloroacetaldehyde; (iv) one or more of the genes from one or more of the clones showing conversion of these substrates into 4-chloro-3-(S)-hydroxy-butyraldehyde (CHBA) and/or 6-chloro-2,4,6-trideoxy-D-erythrohexapyranoside (CTeHP) are isolated and re-cloned into the same genetic background as for [SEQ ID No. 6];
   and wherein
(B) the DERA enzymes encoded by the re-cloned genes obtained in step (iv) are expressed and tested by means of the DERA Productivity Factor Test, thereby obtaining a productivity factor for each of such wild-type enzymes;
   and wherein
(C) the productivity factor for these wild-type enzymes from step (B) is compared to that of the wild-type enzyme from Escherichia coli K12 (EC 4.1.2.4) having a sequence of [SEQ ID No. 1], and one or more genes encoding a DERA enzyme having at least 10% higher productivity factor in the said comparison are selected and isolated.

Isolation of total and/or genomic DNA and/or cDNA, as meant in step (i) above, may be done, for instance, from microorganisms or from environmental samples such as soil or water. The expression library of isolated DNA as prepared in step (ii) consists of individual clones, comprising said isolated DNA, which DNA encodes one or more different enzymes. The incubation with a mixture of acetaldehyde and chloroacetaldehyde in step (iii) above, for the assessment of presence of DERA activity, may be performed with such mixtures in a wide molecular ratio range of these substrates, for instance of from 0.2:1 to 5:1. It will be clear, that already qualitative assessment of the conversion of these substrates into 4-chloro-3-(S)-hydroxy-butyraldehyde (CHBA) and/or 6-chloro-2,4,6-trideoxy-D-erythrohexapyranoside (CTeHP) may provide a first indication of the effectiveness of the genes present in the individual clones from the step (ii) expression library.

Already at this stage, therefore, some ranking in activity of the various genes encoding DERA enzymes can be established. This assessment allows for isolation of the most promising genes. However, since the ultimate aim of the screening process is to find (wild-type) DERAs having a productivity factor, as determined by the DPFT, in the production of 6-chloro-2,4,6-trideoxy-D-erythrohexapyranoside (CTeHP) from an at least equimolar mixture of acetaldehyde and chloroacetaldehyde, which is at least 10% higher than the productivity factor for the 2-deoxy-D-ribose 5-phosphate aldolase enzyme from Escherichia coli K12, these selected genes, or a smaller number thereof as desired, are isolated and re-cloned into the same genetic background as for [SEQ ID No. 6]. This step ensures proper expression of the enzymes to be tested in a comparable way with the expression of the wild-type DERA enzyme from Escherichia coli K12. After screening and testing by means of the DPFT, and making the proper comparison with the results of the DPFT for the wild-type DERA enzyme from Escherichia coli K12, it is very easy to find suitable wild-type DERAs, for instance such DERAs as then can be used as starting point for obtaining mutants according to the present invention.

The invention, moreover, relates to a process for the screening for mutant enzymes from the group of 2-deoxy-D-ribose 5-phosphate aldolase enzymes having a productivity factor, as determined by the DERA Productivity Factor Test, in the production of 6-chloro-2,4,6-trideoxy-D-erythrohexapyranoside (CTeHP) from an at least equimolar mixture of acetaldehyde and chloroacetaldehyde, which is either at least 10% higher than the productivity factor for the corresponding wild-type enzyme or is at least 10% higher than the productivity factor for the 2-deoxy-D-ribose 5-phosphate aldolase enzyme from Escherichia coli K12 (EC 4.1.2.4) having a wild-type enzyme sequence of [SEQ ID No. 1]. In said process (A) subsequently (i) genes encoding a wild-type 2-deoxy-D-ribose 5-phosphate aldolase enzyme are mutated and cloned, in a manner known per se, into the same genetic background as for the gene encoding E. coli K12 DERA having [SEQ ID No. 6], respectively into the same genetic background as for the corresponding wild-type gene from which it is a mutant, thereby obtaining an expression library of clones from the mutants thus prepared; and wherein (B) the DERA enzymes in the clones are expressed and tested by means of the DERA Productivity Factor Test, thereby obtaining a productivity factor for each of the mutant enzymes; and wherein (C) the productivity factor for the mutant enzymes is compared to that for the corresponding wild-type enzyme, or to that of the wild-type enzyme from Escherichia coli K12 (EC 4.1.2.4) having a sequence of [SEQ ID No. 1], and one or more genes encoding a DERA mutant having at least 10% higher productivity factor in the respective comparison are selected and isolated.

More in particular, the invention relates to a process wherein (A) subsequently (i) genes encoding a wild-type 2-deoxy-D-ribose 5-phosphate aldolase enzyme are mutated and cloned, in a manner known per se, into the same genetic background as for E. coli K12 DERA, respectively for the corresponding wild-type gene from which it is a mutant, thereby obtaining an expression library of clones from the mutants thus prepared; (ii) the individual clones from the obtained expression library are incubated with a mixture of the substrates acetaldehyde and chloroacetaldehyde; (iii) one or more of the clones showing highest conversion of these substrates into 4-chloro-3-(S)-hydroxy-butyraldehyde (CHBA) and/or 6-chloro-2,4,6-trideoxy-D-erythrohexapyranoside (CTeHP) are selected; (B) the DERA enzymes in the selected clones from step (iii) are expressed and tested by means of the DERA Productivity Factor Test, thereby obtaining a productivity factor for each of the mutant enzymes; and (C) the productivity factor for the screened mutant enzymes is compared to that for the corresponding wild-type enzyme, or to that of the wild-type enzyme from Escherichia coli K12 (EC 4.1.2.4) having a sequence of [SEQ ID No. 1], and one or more genes encoding a DERA mutant having at least 10% higher productivity factor in the respective comparison are selected and isolated.

This second type of screening, for mutants, starts from genes known to be encoding a wild-type 2-deoxy-D-ribose 5-phosphate aldolase enzyme for example obtained using the process for the screening for wild-type DERA enzymes according to the invention or from genes encoding wild-type DERA enzymes e.g. as referenced in table 1 or 2. These genes first are mutated and cloned, in a manner known per se, into the same genetic background as for E. coli K12 DERA, respectively for the corresponding wild-type gene from which it is a mutant. Said genes, for instance, may be obtained from microorganisms or from environmental samples such as soil or water. The aforementioned mutating and cloning results in an expression library of clones from the mutants thus prepared. In fact, as is well-known to the skilled man, such expression library is prepared by subsequently preparing a DNA library of the mutants, cloning each of the individual DNAs into a vector, and transforming the vectors into a suitable expression host. The incubation with a mixture of acetaldehyde and chloroacetaldehyde in step (ii) above, for the assessment of presence of DERA activity, again may be performed with such mixtures in a wide molecular ratio range of these substrates, for instance of from 0.2:1 to 5:1. The qualitative assessment of the conversion of these substrates into 4-chloro-3-(S)-hydroxy-butyraldehyde (CHBA) and/or 6-chloro-2,4,6-trideoxy-D-erythrohexapyranoside (CTeHP) then results in a first ranking of the degree of conversion of these substrates into 4-chloro-3-(S)-hydroxy-butyraldehyde (CHBA) and/or 6-chloro-2,4,6-trideoxy-D-erythrohexapyranoside (CTeHP), and one or more of the clones showing highest conversion may be selected for further evaluation by means of the DPFT. It is needless to say, that proper expression of the enzymes to be tested should be ensured in order that the test results can be readily compared with those for the expression of the wild-type DERA enzyme from Escherichia coli K12, respectively for the corresponding wild-type gene from which it is a mutant. In this way it is very easy to find and isolate suitable genes encoding mutant DERAs, as then suitably can be used in the commercial production of valuable pharmaceutical products such as statins.

It is to be noticed that the above described screening process is different from the one used by W. A. Greenberg et al., in PNAS, vol. 101, p. 5788-5793 (2004), cited above. The authors of said article namely used a fluorescent detection assay, as has been described by R. Pérez Carlón et al. in Chem. Eur. J., 6, p. 4154-4162 (2000). Said detection assay is a very indirect method wherein the DERA activity is being determined by means of a fluorescent umbelliferone derivative of the 2-deoxy-D-ribose substrate. Said method, however, is less suitable (because requiring an additional assay for determining the desired activity in the desired reaction with substituted aldehydes) for the determination of DERA productivity (as well as activity) in the production of 6-chloro-2,4,6-trideoxy-D-erythrohexapyranoside (CTeHP) from an at least equimolar mixture of acetaldehyde and chloroacetaldehyde, because in the first instance only enzymes are obtained, which display a retroaldol reaction very similar to the DERA natural substrate reaction and those are tested for the target reaction in an additional, second screening. To overcome such problems, the present inventors have developed their own, direct, screening method and also developed the so-called DERA Productivity Factor Test.

Suitably, in said screening for mutants in the first step genes encoding a wild-type 2-deoxy-D-ribose 5-phosphate aldolase enzyme are mutated, that originate from one of the sources indicated in the tables 1, 2 and 3.

The present invention accordingly also relates to isolated nucleic acids obtainable by any of such screening processes, in particular as are obtainable by the screening process applied to mutated genes encoding a wild-type 2-deoxy-D-ribose 5-phosphate aldolase enzyme, that originate from one of the sources indicated in the tables 1, 2 and 3.

The present invention further relates to an isolated nucleic acid encoding a mutant 2-deoxy-D-ribose 5-phosphate aldolase enzyme, wherein the isolated nucleic acid encodes for a mutant having a productivity factor which is at least 10% higher than the productivity factor for the corresponding wild-type enzyme from which it is a mutant and wherein the productivity factors of both the mutant and the corresponding wild-type enzyme are measured under identical conditions.

Moreover, the present invention relates to an isolated nucleic acid encoding a mutant 2-deoxy-D-ribose 5-phosphate aldolase enzyme, wherein the isolated nucleic acid encodes for a mutant having a productivity factor which is at least 10% higher than the productivity factor for the corresponding wild-type enzyme from which it is a mutant and wherein the productivity factors of both the mutant and the corresponding wild-type enzyme are measured under identical conditions and having a productivity factor which is at least 10% higher than the productivity factor for the 2-deoxy-D-ribose 5-phosphate aldolase from Escherichia coli K12 (EC 4.1.2.4) having the wild-type enzyme sequence of [SEQ ID No. 1] and wherein the productivity factors of both the mutant and the Escherichia coli K12 enzyme are measured under identical conditions.

Furthermore, the invention also relates to an isolated nucleic acid encoding a mutant from Escherichia coli K12 (EC 4.1.2.4) having the wild-type enzyme sequence of [SEQ ID No. 1]. Moreover, the invention also relates to an isolated nucleic acid encoding a mutant 2-deoxy-D-ribose 5-phosphate aldolase enzyme having at least one amino acid substitution at one or more of the positions, or at one or more of the positions K13, T19, Y49, N80, D84, A93, E127, A128, K146, K160, I166, A174, M185, K196, F200, and S239 in [SEQ ID No. 1] or at positions corresponding thereto, preferably at the position F200 or at a position corresponding thereto, and/or a deletion of at least one amino acid at one of the positions S258 or Y259 in [SEQ ID No. 1] or at positions corresponding thereto, optionally in combination with C-terminal extension, preferably by one of the fragments TTKTQLSCTKW [SEQ ID No. 2] and KTQLSCTKW [SEQ ID No. 3] and/or in combination with an N-terminal extension. Preferably, the said isolated nucleic acid encodes an mutant 2-deoxy-D-ribose 5-phosphate aldolase enzyme having at least one of the amino acid substitutions in, or corresponding to the substitutions in, [SEQ ID No. 1] selected from the group consisting of:
a. K13 and/or K196 replaced by a positively charged amino acid, preferably by R or H;
b. T19 and/or M185 replaced by another amino acid, preferably by another amino acid selected from the groups consisting of hydrophilic amino acids, in particular consisting of S, T, C, Q, and N, and/or hydrophobic amino acids, in particular consisting of V, L and I;
c. Y49 replaced by an aromatic amino acid selected from the group consisting of F and W;
d. N80 and/or I166 and/or S239 replaced by another amino acid selected from the group of hydrophilic amino acids consisting of T, S, C, Q and N;
e. D84 and/or A93 and/or E127 replaced by another, preferably smaller, amino acid selected from the group of small amino acids consisting of, in order of decreasing size, E, T, N, P, D, C, S, A, and G;
f. A128 and/or K146 and/or K160 and/or A174 and/or F200 replaced by another amino acid selected from the group of hydrophobic amino acids consisting of I, L, M, V, F, and Y;
   and/or having a deletion of at least one amino acid at the positions S258 and Y259 in [SEQ ID No. 1], or at positions corresponding thereto, optionally in combination with C-terminal extension, preferably by one of the fragments TTKTQLSCTKW [SEQ ID No. 2] and KTQLSCTKW [SEQ ID No. 3] and/or in combination with N-terminal extension.

Most preferably, the isolated nucleic acid according to the present invention encodes a mutant 2-deoxy-D-ribose 5-phosphate aldolase enzyme having at least one or more of the mutations in, or corresponding to the mutations in, [SEQ ID No. 1] selected from the group of K13R, T19S, Y49F, N80S, D84G, A93G, E127G, A128V, K146V, K160M, I166T, A174V, M185T, M185V, K196R, F2001, F200V, F200M and S239C, and/or a deletion of at least one amino acid at the positions ΔS258 and ΔY259 in [SEQ ID No. 1], or at positions corresponding thereto, optionally in combination with C-terminal extension by one of the fragments TTKTQLSCTKW [SEQ ID No. 2] and KTQLSCTKW [SEQ ID No. 3].

More in particular, the nucleic acid according to the present invention encodes a mutant 2-deoxy-D-ribose 5-phosphate aldolase enzyme having at least the following two mutations in, or corresponding to the two mutations in, [SEQ ID No. 1] selected from the group of F2001 and ΔY259; F200M and ΔY259; F200V and ΔY259; F200I and C-terminal extension by KTQLSCTKW [SEQ ID No. 3]; F200M and C-terminal extension by KTQLSCTKW [SEQ ID No. 3]; and F200V and C-terminal extension by KTQLSCTKW [SEQ ID No. 3];

Further, the invention relates to vectors comprising any of such nucleic acids as described hereinabove, as well as to host cells comprising a mutant from the group of 2-deoxy-D-ribose 5-phosphate aldolase wild-type enzymes as described in the foregoing, or to such mutant enzymes obtainable according to the screening processes as described hereinabove, and/or to host cells comprising an isolated nucleic acid as described in the foregoing and/or comprising such vectors as described before.

The present invention equally relates to a process for the preparation of mutant 2-deoxy-D-ribose 5-phosphate aldolases having a productivity factor which is at least 10% higher than the productivity factor for the corresponding wild-type enzyme and/or for the 2-deoxy-D-ribose 5-phosphate aldolase enzyme from Escherichia coli (EC 4.1.2.4) having a wild-type enzyme sequence of [SEQ ID No. 1], wherein use is made of nucleic acids as described hereinabove, or of vectors as described hereinabove, or of host cells as described hereinabove.

The present invention also relates to an improved process for the preparation of a 2,4-dideoxyhexose or a 2,4,6-trideoxyhexose of formula 1

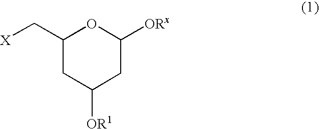

wherein R¹ and R^{x} each independently stand for H or a protecting group and wherein X stands for a halogen; a tosylate group; a mesylate group; an acyloxy group; a phenylacetyloxy group; an alkoxy group or an aryloxy group from acetaldehyde and the corresponding substituted acetaldehyde of formula HC(O)CH₂X, wherein X is as defined above, wherein a mutant DERA enzyme according to the present invention, or produced by a process according to the present invention, or obtainable by the process for screening of mutant enzymes according to the present invention, is used, and wherein—in case R¹ and/or R^{x} stand for a protecting group, the hydroxy group(s) in the formed compound is/are protected by the protecting group in a manner known per se.

Preferably, X stands for a halogen, more preferably Cl, Br or I; or for an acyloxy group, more preferably an acetoxy group.

The mutant DERA enzyme may be employed in the above described reaction using reaction conditions as described in the art for these reactions using wild type DERA enzymes, for instance using the reaction conditions as described in U.S. Pat. No. 5,795,749, for instance in column 4, lines 1-18 or for instance using fed-batch reaction conditions as described in W. A. Greenberg et al., PNAS, vol. 101, pp 5788-5793, (2004).

Preferably, the mutant DERA enzyme of the invention is employed in the above described reaction using reaction conditions as described in WO03/006656: The carbonyl concentration, that is the sum of the concentration of aldehyde, 2-substituted aldehyde and the intermediate product formed in the reaction between the aldehyde and the 2-substituted aldehyde (namely a 4-substituted-3-hydroxy-butyraldehyde intermediate), is preferably held at a value below 6 moles/l during the synthesis process. It will be clear to one skilled in the art that slightly higher concentration for a (very) short time will have little effect. More preferably, the carbonyl concentration is chosen between 0.1 and 5 moles per liter of reaction mixture, most preferably between 0.6 and 4 moles per liter of reaction mixture.

The reaction temperature and the pH are not critical and both are chosen as a function of the substrate. Preferably the reaction is carried out in the liquid phase. The reaction can be carried out for example at a reaction temperature between −5 and +45° C., and at a pH between 5.5 and 9, preferably between 6 and 8.

The reaction is preferably carried out at more or less constant pH, use for example being made of a buffer or of automatic titration. As a buffer for example sodium and potassium bicarbonate, sodium and potassium phosphate, triethanolamine/HCl, bis-tris-propane/HCl and HEPES/KOH can be applied. Preferably a potassium or sodium bicarbonate buffer is applied, for example in a concentration between 20 and 400 mmoles/l of reaction mixture.

The molar ratio between the total quantity of aldehyde and the total quantity of 2-substituted aldehyde is not very critical and preferably lies between 1.5:1 and 4:1, in particular between 1.8:1 and 2.2:1.

The amount of mutant DERA enzyme used in the process of the invention is in principle not critical. It is routine experimentation to determine the optimal concentration of enzyme for an enzymatic reaction and so the person skilled in the art can easily determine the amount of mutant DERA enzyme to be used.

In a preferred embodiment of the invention, R¹ and R^{x} both stand for H. In an even more preferred embodiment of the invention, the compound of formula (1) is enantiomerically enriched.

Protecting groups which may be represented by R¹ and R^{X} include alcohol protecting groups, examples of which are well known in the part. Particular example include tetrahydropyranyl groups. Preferred protecting groups are silyl groups, for example triaryl- and preferably trialkylsilyl group and hydrocarbyl groups. Even more preferred protecting groups are benzyl, methyl, trimethylsilyl, t-butylmethylsilyl and t-butyldiphenylsilyl groups.

Protecting groups which may be represented by R¹ and R^{x} may be the same or different. When the protecting groups R¹ and R^{x} are different, advantageously this may allow for selective removal of only R¹ and R^{x}. Preferably, when the protecting groups R¹ and R^{x} are different, R¹ is a benzyl or silyl group and R^{x} is a methyl group.

The compound of formula (1), wherein R^{x} stands for H, may be used in a process (analogous to the process) as described in WO04/096788, WO05/012246 or WO04/027075. Therefore, the invention also relates to a process, wherein the compound of formula (1), wherein X and R¹ are as defined above and wherein R^{x} stands for H is produced according to the invention and is subsequently reacted with an oxidizing agent to form the corresponding compound of formula (2)

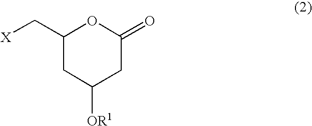

wherein X and R¹ are as defined above and which compound of formula 2 is subsequently reacted with a cyanide ion to form a compound of formula (3)

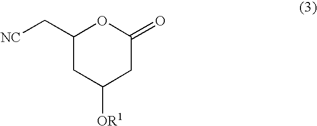

wherein R¹ is as defined above.

For this reaction use may be made of the process conditions as described for this process step in WO04/096788 on page 2, line 10-page 3, line 13. Alternatively, the process conditions as described in WO 05/012246 (see e.g. page 5, lines 19-26) or as described in WO 04/027075 (for example described in example 2) may be used.

In a different embodiment of the invention, the compound of formula (1) may first be reacted with a cyanide ion, for example under the process conditions as described in WO 05/012246 or using the process conditions of WO04/096788 or of WO 04/027075, to form a compound of formula (4)

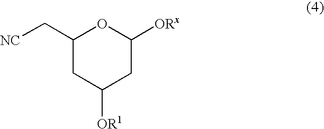

wherein R¹ and R^{x} each independently stand for H or a protecting group, after which the compound of formula (4),—in case R^{x} stands for a protecting group after removal of the protecting group R^{x}—, may be reacted with an oxidizing agent to form the corresponding compound of formula (3), wherein R¹ is as defined above.

For the above cyanation reactions, water may be used as a solvent in combination with other solvents, for example with tetrahydrofuran, CH₃CN, alcohols, dioxane, dimethylsulfoxide, dimethylformamide, N-methylpyrrolidone, toluene, diethylether and/or methyl-t-butyl ether. Preferably at least 5% w/w, more preferably at least 10% w/w, even more preferably at least 20% w/w, even more preferably at least 30% w/w, even more preferably at least 40% w/w, even more preferably at least 50% w/w, even more preferably at least 60% w/w, even more preferably at least 70% w/w, even more preferably at least 80% w/w water, most preferably at least 90% w/w of water in other solvent is used. For practical reasons, it is in particular preferred to use water as the only solvent.

Using the process and reaction conditions as described in WO04/096788 (e.g. on page 5, line 14-page 7, line 3), the compound of formula (4) may be subsequently converted into a compound of formula (5)

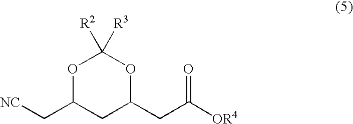

wherein R², R³ and R⁴ each independently stand for an alkyl with for instance 1 to 12 C-atoms, preferably 1-6 C-atoms, an alkenyl with for instance 1 to 12 C-atoms, preferably 1-6 C-atoms, a cycloalkyl with for instance 3-7 C-atoms, a cycloalkenyl with for instance 3-7 C-atoms, an aryl with for instance 6-10 C-atoms or an aralkyl with for instance 7 to 12 C-atoms, each of R², R³ and R⁴ may be substituted and wherein R² and R³ may form a ring together with the C-atom to which they are bound, use being made of a suitable acetal forming agent, in the presence of an acid catalyst, for example as described in WO 02/06266.

According to WO 04/096788, the compound of formula 5, wherein R², R³ and R⁴ are as defined above may be subsequently hydrolysed to form the corresponding salt of formula 6,

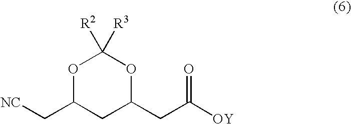

wherein Y stands for an alkali metal, for instance lithium, sodium, potassium, preferably sodium; an alkali earth metal, for instance magnesium or calcium, preferably calcium; or a substituted or unsubstituted ammonium group, preferably a tetraalkyl ammonium group, for example as described in WO04/096788 on page 7, line 4-page 8, line 16). Optionally, the hydrolysis is followed by conversion to the corresponding compound of formula (6), wherein Y is H, for example as described in WO 02/06266.

According to WO 04/096788, the salt of formula (6) may further be converted into the corresponding ester of formula 7

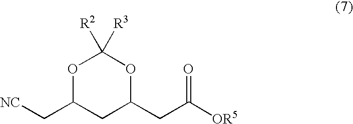

wherein R² and R³ are as defined above and wherein R⁵ may represent the same groups as given above for R² and R³, in a manner known per se (for example as described in WO 02/06266).

For example R⁵ may represent a methyl, ethyl, propyl, isobutyl or tert butyl group. An important group of esters of formula 8 that can be prepared with the process according to the invention are tert butyl esters (R⁵ represents tert butyl).

In a special aspect of the invention the salt of formula (6) is converted into the corresponding ester of formula (7) by contacting the salt of formula (6) in an inert solvent, for example toluene, with an acid chloride forming agent to form the corresponding acid chloride and by contacting the formed acid chloride with an alcohol of formula R⁵OH, wherein R⁵ is as defined above, in the presence of N-methyl morpholine (NMM) according to the process described in WO03/106447 and in WO04/096788, page 9, line 2-page 10, line 2.

The compounds prepared using the process of the invention are particularly useful in the preparation of an active ingredient of a pharmaceutical preparation, for example in the preparation of HMG-CoA reductase inhibitors, more in particular in the preparation of statines, for example, lovastatine, cerivastatine, rosuvastatine, simvastatine, pravastatine and fluvastatine, in particular for ZD-4522 as described in Drugs of the future (1999), 24(5), 511-513 by M. Watanabe et al., Bioorg & Med. Chem. (1997), 5(2), 437-444. The invention therefore provides a new, economically attractive route for the preparation of compounds, in particular the compound of formula (1), that can be used for the synthesis of statines. A particularly interesting example of such a preparation is the preparation of Atorvastatin calcium as described by A. Kleemann, J. Engel; pharmaceutical substances, synthesis, patents, applications 4th edition, 2001 Georg Thieme Verlag, p. 146-150.

Therefore, the invention also relates to a process, wherein a compound obtained in a process according to the invention is further converted into a statin, preferably atorvastatin or a salt thereof, for instance its calcium salt, using the process of the invention and further process steps known per se. Such processes are well known in the art.

The invention will now be explained by means of the following experimental results without being restricted thereto in any way.

### EXPERIMENTAL

### General Part

Methods to Identify DERA Mutants with Improved Resistance or Productivity.

Two methods to identify DERA mutants with improved resistance or productivity can be used One method examines the resistance of DERA mutants towards chloroacetaldehyde, the other assesses the productivity of DERA mutants in the production of 6-chloro-2,4,6-trideoxy-D-erythrohexapyranoside (CTeHP) using chloroacetaldehyde and acetaldehyde as substrates. The first method examines the resistance of DERA mutants to chloroacetaldehyde using a microtiter based form of the standard DERA natural substrate activity assay, using the natural DERA substrate 2-deoxy-D-ribose 5-phosphate as substrate. The second method analyzes the productivity of DERA mutants on acetaldehyde and chloroacetaldehyde as substrates in the production of 4-Chloro-3-(S)-hydroxy-butyraldehyde (CHBA), which is the product of the DERA catalyzed aldol reaction with one molecule each of acetaldehyde and chloroacetaldehyde and therefore an intermediate in the reaction to CTeHP, using a high through-put gas chromatography coupled to mass spectroscopy (GC/MS) analysis method.

### Determination Protein Concentrations in Solution

The concentrations of proteins in solutions such as cell-free extracts (cfe) were determined using a modified protein-dye binding method as described by Bradford in Anal. Biochem. 72: 248-254 (1976). Of each sample 50 μl in an appropriate dilution was incubated with 950 μl reagent (100 mg Brilliant Blue G250 dissolved in 46 ml ethanol and 100 ml 85% ortho-phosphoric acid, filled up to 1,000 ml with milli-Q water) for at least five minutes at room temperature. The absorption of each sample at a wavelength of 595 nm was measured in a Perkin Elmer Lambda20 UV/VIS spectrometer. Using a calibration line determined with solutions containing known concentrations of bovine serum albumin (BSA, ranging from 0.025 mg/ml to 0.25 mg/ml) the protein concentration in the samples was calculated.

### DERA Productivity Factor Test

Selected clones from both methods, which show improved resistance to chloroacetaldehyde or increased CHBA formation can be characterized with respect to their productivity in the formation of CTeHP using the DERA Productivity Factor Test. For this characterization a volume of cfe which contains between 1.0 and 1.4 mg of cfe is incubated with 0.04 mmol chloroacetaldehyde and 0.093 mmol acetaldehyde in 0.1 M NaHCO₃ buffer (final pH=7.2) in a total volume of 0.2 ml with stirring. After 16 h the reactions are stopped by addition of 9 volumes of acetone or acetonitrile and centrifuged for 10 minutes at 16.000×g. The supernatant is analyzed by gas chromatography on a Chrompack CP-SIL8CB column (Varian) using a FID detector for their CTeHP and CHBA content. The amount of CTeHP in mmol formed by 1 mg of cell-free extract proteins containing wild-type or mutated DERA within 16 hours at pH 7.2 at room temperature (25° C.) at substrate concentrations of 0.2 M chloroacetaldehyde and 0.4 M acetaldehyde is defined as “DERA Productivity Factor”.

### DERA Natural Substrate Activity Assay

For the estimation of DERA activity the initial activity in the DERA natural substrate reaction, the aldol cleavage of 2-deoxy-D-ribose 5-phosphate to acetaldehyde and D-glyceraldehyde 3-phosphate, can be determined at room temperature (RT). 10 μl cell-free extract is transferred into 140 μl of 50 mM triethanolamine buffer (pH 7,5). The activity assay is started by adding 50 μl of auxiliary enzyme and substrate mix solution (0.8 mM NADH, 2 mM 2-deoxy-D-ribose 5-phosphate, triose phosphate isomerase (30 U/ml, Roche Diagnostics) and glycerol phosphate dehydrogenase (10 U/ml, Roche Diagnostics)). The reaction is stopped after 30 seconds by adding 50 μl Stop solution (6 M guanidine hydrochloride, 100 mM sodium hydrogenphosphate, 10 mM TrisHCl pH 7.5). The initial DERA activity present is determined by measuring the UV-absorbance of the sample at 340 nm wavelength. The consumption of one molecule of NADH corresponds to the cleavage of one molecule of 2-deoxy-D-ribose 5-phosphate.

### Example 1

### DERA Mutants with Improved Resistance for Chloroacetaldehyde

Construction of E. Coli Variant deoC Library by Random Mutagenesis.

For the construction of a random mutagenesis library of the E. coli K12 deoC gene [SEQ ID No. 6], which codes for the E. coli K12 DERA enzyme [SEQ ID No. 1], the Clonetech Diversify PCR Random Mutagenesis Kit was used. Several reactions with varying MnSO₄ concentration (whereby more mutations are being introduced as such concentration is higher) were performed according to the supplier's manual resulting in 1 to 3 point mutations into the Escherichia coli K12 deoC gene, resulting in 1 to 2 amino acid exchanges in the DERA enzyme amino acid sequence. For the amplification of the E. coli deoC gene [SEQ ID No. 6], encoding the E. coli 2-deoxy-D-ribose 5-phosphate aldolase [SEQ ID No. 1], the primers DAI 13600 and DAI 13465 (corresponding to [SEQ ID No. 4] and [SEQ ID No. 5], respectively) were used as forward and reverse primer, respectively. Both primers contained sites compatible for cloning the obtained PCR amplified deoC gene fragment via site-specific recombination, using Gateway Technology (Invitrogen).

Sequence of forward primer (DAI 13600):

[SEQ ID No. 4]

5′ GGG GAC AAG TTT GTA CAA AAA AGC AGG CTT CGA AGG
AGA TAG AAC CAT GAC TGA TCT GAA AGC AAG CAG CC 3′
Sequence of reverse primer (DAI 13465):

[SEQ ID No. 5]

5′ GGG GAC CAC TTT GTA CAA GAA AGC TGG GTC TTA GTA
GCT GCT GGC GCT C 3′

The error-prone PCR amplification used the following temperature program; 94° C. for 2 minutes, 25 cycles with 94° C. for 30 seconds and 68° C. for 1 minute, followed by 68° C. for 10 minutes. Error-prone PCR fragments were first cloned into a pDONR (Invitrogen) vector and large-scale pENTR clone plasmid preparations were made starting with more than 20,000 colonies. These pENTR preparations were then used for the construction of expression constructs using the pDEST14 vector (Invitrogen). Expression constructs were then transformed into chemically competent E. coli BL21 Star (DE3) for expression of the mutated E. coli K12 deoC gene coding for DERA enzyme mutants.

Expression of Mutated deoC Genes in Deep-Well Microtiter Plates

Colonies were picked from Q-trays using the Genetix Q-pics and 200 μl 2*TY medium (containing 100 μg/ml ampicillin) cultures in microtiter plates (MTP) were inoculated, these pre-cultures were then grown on a gyratory shaker either at 25° C. for 2 days, or at 37° C. overnight. From the pre-cultures 100 μl were used to inoculate 500 μl expression cultures (2*TY, 100 μg/ml ampicillin, 1 mM IPTG) in deep-well plates; these expression cultures were then grown on a gyratory shaker at 37° C. for 24 hours.

### Microtiter Plate DERA Stability Assay

For the examination of the resistance of mutated DERA enzymes towards chloroacetaldehyde an assay can be employed, which is based on the DERA natural substrate reaction. The deep-well expression cultures are centrifuged at 4,000 rotations per minute (rpm) for 15 minutes and the obtained E. coli cell pellets are lysed in 400 μl of B-PER lysis buffer (25% v/v B-PERII (Pierce), 75% (v/v) 50 mM triethanolamine buffer, pH 7.5 plus 100 mg/l RNAse A). For chloroacetaldehyde concentrations above 120 mM chloroacetaldehyde, 200 mM triethanolamine is used. Cell debris is removed by centrifugation (4,000 rpm, 4° C. for 15 minutes) and 210 μl cell-free extract from each well is transferred into a new microtiter plate. For the estimation of DERA activity the initial activity in the DERA natural substrate reaction is determined using the DERA Natural Substrate Activity Assay as described above. The resistance of the DERA mutants to chloroacetaldehyde is examined by taking the remaining 200 μl volume of cell-free extract and adding 50 μl of chloroacetaldehyde solution.

In the first screening round a chloroacetaldehyde stock solution of 600 mM, for screening the first recombined mutant library a 1.0 M stock, and for the second recombined mutant library a 1.5 M stock, was used, resulting in final concentrations of 120, 200, and 300 mM of chloroacetaldehyde, respectively. In all cases the exposure time was 2 minutes. Thereafter 50 μl samples (error-prone PCR library), 30 μl samples (first recombined mutant library) or 25 μl sample (second recombined mutant library), respectively, were taken and transferred to a microtiter plate containing 50 mM triethanolamine buffer (pH 7.5, final volume of 200 μl). The remaining DERA activity for the DERA natural substrate reaction was determined, similar to initial DERA activity, by adding 50 μl of the auxiliary-enzyme/substrate mix. The DERA natural reaction assay was allowed to proceed for 30 seconds before 50 μl of Stop solution was added. To determine the amount of consumed NADH, the UV-absorbance of the samples were measured at 340 nm.

### Recombination of Favorable Mutations Using Blunt-End Restriction Enzyme (BERE) Recombination (According to WO03/010311)

Mutant clones, selected from the error-prone PCR library, were used as a basis for further improvement of DERA by recombination of their mutations. Plasmid DNA of selected mutant clones was isolated from stock cultures and used as template to amplify the mutated genes. The resulting mutant gene PCR fragments were digested with blunt end cutting restriction endonucleases, the obtained gene fragments were reassembled into full-length genes using ampligase and Hercules DNA polymerase. For the recombination two gene fragment pools were made using the restriction endonuclease HaeIII, HinCII and FspI (pool A) and CacI8 or BstUI (pool B). For the ampligase reaction (50 μl total volume), with 0.5 μg of gene fragment DNA from each pool, the following temperature program was used: 94° C. for 2 minutes, 30 cycles of 94° C. for 30 seconds and 60° C. for 1 minute, and a final 60° C. cycle of 10 minutes. 20 μl of the ampligase reaction were ethanol precipitated, the DNA pellet (about 0.4 μg DNA) was dissolved in 40 μl sterile water and used as template for PCR amplification of the recombined mutant genes. For the PCR reaction (50 μl volume) using Hercules DNA polymerase (5 U) primer DAI 13600 ([SEQ ID No. 4]) and DAI 13465 ([SEQ ID No. 5]) were used as forward and reverse primers, respectively. The following PCR program was used: 72° C. for 5 minutes, 15 cycles of 94° C. for 30 seconds, 50° C. for 30 seconds, and 72° C. for 45 seconds, final cycle 72° C. for 10 minutes. The obtained full-length mutant gene fragments were purified, using the Qiagen PCR purification kit, and cloned into pDEST14 vector using site-specific recombination as described above. Re-examination of DERA mutants with improved chloroacetaldehyde resistance

DERA enzyme mutants pre-cultures were inoculated from the frozen glycerol master plate and incubated overnight with shaking at 180 rpm and at 25° C. Pre-culture aliquots were used to inoculate 25 ml expression cultures (2*TY medium, 100 μg/ml ampicillin, 1 mM IPTG) and incubated for 36 hours at 25° C. (shaking with 180 rpm). Cells were harvested by centrifugation (5,000 rpm, 15 minutes) and the cell pellet lysed using 2.5 ml of B-PER II. Cell debris was removed by centrifugation first for 15 minutes at 5,000 rpm, then using an Eppendorf benchtop centrifuge for 15 min at 14,000 rpm (4° C.). The obtained cell-free extracts were used to examine the resistance of the expressed DERA mutant enzymes towards chloroacetaldehyde in time course experiments and over concentration ranges.

For the time course experiments the initial DERA natural substrate reaction activity present in the sample was determined in quadruplicates. A defined volume of extract with a suitable amount of DERA activity was exposed to 200 mM of chloroacetaldehyde and at time points t=1, t=5, t=10, t=15, and t=20 minutes after chloroacetaldehyde addition, aliquots were withdrawn and the remaining amount of DERA activity measured, using the DERA natural substrate activity assay in quadruplicates. The determined initial DERA natural substrate activity was set as 100% and the activities determined at the indicated time points were expressed as percentage relative to the said initial starting DERA natural substrate activity.

### Results of the Chloroacetaldehyde Resistance Method

Using the above described resistance method about 10,000 clones were examined. In the initial stability campaign, the error-prone PCR derived mutants, the DERA enzymes were exposed to 150 mM chloroacetaldehyde for 2 minutes. For the screening of the recombined variants the concentration of chloroacetaldehyde was increased to 200 mM in the first recombination and 300 mM in the second recombination round, respectively. Selected mutant clone were re-investigated in triplicates using the same setup. Clones performing similar to the initial results were selected and isolated.

The pooled mutated deoC genes of these selected clones were randomly recombined using the BERE-method (as described above). In the first recombination round 1,000 clones were investigated at 200 mM chloroacetaldehyde. 22 clones were isolated, which exhibited an at least 50 percent increased resistance against chloroacetaldehyde. These mutant clones were again isolated from the master plates, expression vectors purified, mutated genes amplified by PCR, and pooled. In the second recombination round 41 DERA enzyme mutants, that showed an at least two times increased resistance at 300 mM chloroacetaldehyde compared to the E. coli K12 wild-type DERA after 2 minutes incubation time, were identified.

The 10 best mutants of the second round were re-tested from 25 ml expression cultures for their resistance to 200 mM chloroacetaldehyde in parallel to the E. coli K12 wild-type DERA applying the DERA natural substrate reaction activity assay. The results are the mean of three independent experiments and given as percent residual DERA activity compared to the respective values at 0 mM chloroacetaldehyde in table 4 including the designation and the amino acid exchanges of the DERA enzyme mutants.

TABLE 4
Resistance to chloroacetaldehyde and DERA Productivity
Factor of Escherichia coli K12 DERA enzyme mutants and the
E. coli K12 wild-type DERA

| | | residual activity | DERA |
|---|---|---|---|
| | | [in %] at 0.2 M | Productivity |
| clone | amino acid exchange(s) | chloroacetaldehyde | Factor |
| wild- | — | 26.1 | 3.2 |
| type | | | |
| 13-2H | Y49F | 78.8 | 4.2 |
| 17-2D | ΔY259 | 83.8 | 9.9 |
| 8-6D | K196R, ΔS258, ΔY259, | 152.1 | 5.6 |
| | extension [SEQ ID No. 2] | | |
| 22-2C | Y49F, K160M, M185T | 64.3 | 5.3 |
| 2-3H | K146V, ΔY259 | 364.8 | 7.6 |
| 5-12H | M185V | 58.3 | 15.1 |
| 19-3B | Y49F, M185T | 49.8 | 4.2 |
| 25-10H | Y49F, A128V | 31.4 | 3.8 |
| 25-1D | D84G, ΔS258, ΔY259, | 33.9 | 4.5 |
| | extension [SEQ ID No. 2] | | |
| 21-10F | Q80S, E127G, M185V, | 251.0 | 6.2 |
| | extension [SEQ ID No. 3] | | |

### Example 2

### DERA Mutants Enzymes with Improved Productivity for CHBA

For the screening of DERA mutants with increased productivity of 4-chloro-3-(S)-hydroxy-butyraldehyde (CHBA) formed by aldolization of one molecule of each acetaldehyde and chloroacetaldehyde, a library of about 3,000 mutant clones was constructed. Error-prone PCR, Gateway cloning, and expression of DERA mutants was carried out as described in example 1, except that the error prone PCR fragments were directly cloned into the pDEST14 vector without isolation of pENTR vectors, to maximize the genetic diversity of the expression library.

Sample Preparation for Productivity Method with GC/MS.

For the GC/MS based productivity method examining the CHBA product formation using 200 mM of chloroacetaldehyde and acetaldehyde as substrates, cell-free extracts can be prepared from 600 μl expression cultures, similar to the chloroacetaldehyde resistance screening. Expression cultures which have been incubated in deep-well plates on a gyratory shaker for 24 hours are centrifuged (4000 rpm for 15 minutes). The obtained cell pellets are lysed in 350 μl of 50% (v/v) B-PER II, 50% (v/v) 250 mM NaCO₃, pH 7.5. Cell debris is removed by centrifugation as above. 100 μl of the cfes containing the mutated E. coli K12 DERA enzymes are mixed with 100 μl of a 400 mM solution of both acetaldehyde and chloroacetaldehyde. After 1 hour incubation at RT, 100 μl of each reaction is added to 900 μl of acetonitrile containing 0.05% (w/w) cyclohexylbenzene, which serves as internal standard (IS) for product quantification. Protein precipitate is removed by centrifugation and 500 μl of each sample is transferred to a new deep-well microtiter plate.

Analysis of 4-chloro-3-hydroxy-butyraldehyde by High-Through Put GC/MS

The samples were analyzed for their CHBA content on a Hewlett Packard type 6890 gas chromatograph coupled to a HP 5973 mass detector (Agilent). The samples were injected onto a Chrompack CP-SIL13CB (Varian) column via an automated injector directly from the microtiter plates. A temperature program from 100° C. to 250° C. was performed within two minutes with helium as carrier gas at a constant flow of 1.1 ml/min. Characteristic ions of the internal standard (M=45 from t=0 to 2.80 minutes) and CHBA (M=160 from t=2.80 minutes until end of method) were detected by single ion monitoring (SIM). The total cycle time for one sample (from injection to injection) was below five minutes.

The productivity method delivered 7 enzyme mutants of the E. coli K12 DERA with at least 3 times increased CHBA concentrations compared to the E. coli K12 wild-type DERA. The selected mutant clones were retested using the DERA Productivity Factor Test as described above to compare them with the E. coli K12 wild-type DERA and determine their DERA Productivity Factor (in mmol CTeHP produced per mg protein in the cfe in 16 hours).

2.5 ml Luria Bertani medium (LB) pre-cultures (containing 100 μg/ml carbenicillin) were inoculated with a single colony of every re-transformed mutant clone, and incubated over night with shaking at 180 rotations per minute (rpm) and at 28° C. Out of these pre-cultures 50 ml LB expression cultures containing 100 μg/ml carbenicillin were inoculated to an cell density of OD₆₂₀ₙₘ of 0.05 and cultivated at 28° C. on a gyratory shaker (180 rpm). Expression of the mutant DERAs was induced by addition of 1 mM isopropyl-β-D-thiogalactopyranoside (IPTG) after three hours of incubation and at an optical density of about 0.4. Cells were harvested by centrifugation (5 minutes at 5,000×g) after 21 hours and resuspended in 1 ml of a 50 mM triethanolamine buffer (pH 7.2). The cell-free extract (cfe) was obtained by sonification of the cell suspension for 5 min (10 seconds pulse followed by 10 seconds pause) and centrifugation for one hour at 4° C. and 16,000×g. Cfes were stored at 4° C. until further use in the DERA Productivity Factor Test. The designation and the amino acid exchanges of the DERA enzyme mutants found by the productivity method are listed in table 5.

TABLE 5
CHBA formation and DERA Productivity Factor of Escherichia coli
K12 DERA enzyme mutants and the E. coli K12 wild-type DERA

| | | relative CHBA | DERA |
|---|---|---|---|
| | amino acid | formation [as % | Productivity |
| clone | exchange(s) | wild-type] | Factor |
| wild-type | — | 100 | 3.2 |
| 1-4A | T19I, I166T | 568 | 4.2 |
| 4-4A | K13R | 654 | 8.2 |
| 1-10A | S93G, A174V | 522 | 9.2 |
| 9-11H | F200I | 693 | 44.2 |
| 9-9F | T19S | 373 | 4.8 |
| 15-2F | M185T | 576 | 5.7 |
| 1-11C | S239C | 861 | 5.7 |

### Example 3

### Scale-Up of CTeHP Synthesis with DERA Mutant 9-11H

Chemically competent E. coli BL21 Star (DE3) (Invitrogen) was freshly transformed as described in Example 2 with plasmids pDEST14-Ecol-deoC and pDEST14_{—}9-11H (F200I mutant), respectively. Two 50 ml LB pre-cultures (containing 100 μg/ml carbenicillin) were inoculated with single colonies from the respective transformation agar plates, and incubated over night on a gyratory shaker (180 rpm) at 28° C.

The next day sterile Erlenmeyer flasks containing 1 l LB medium each with 100 μg/ml carbenicillin were inoculated with the 50 ml pre-cultures to a start cell density of OD₆₂₀=0.05 and incubated with shaking (180 rpm) at 28° C. At cell densities of OD₆₂₀=0.6 the expression of wild-type DERA of E. coli K12 and the there from derived mutant DERA 9-11H, containing the amino acid exchange F200I, was induced by addition of 1 mM IPTG. The cultures were further incubated under the same conditions until a total cultivation time of 21 h. At this time point both cultures were harvested by centrifugation (5 minutes at 5000×g) and the cell pellets were resuspended in 25 ml of a 50 mM triethanolamine buffer (pH 7.2). The cell-free extracts were obtained by sonification of the cell suspensions for 2 times 5 minutes (10 seconds pulse followed by 10 seconds pause, large probe) and centrifugation for one hour at 4° C. and 39,000×g. The cfes were kept at 4° C. until further use. The specific activities of both cfes, determined with the DERA Natural Substrate Activity Assay as described above but with 5 mM 2-deoxy-D-ribose 5-phosphate, were in the same range.

For the scaled-up reactions 10 mmol chloroacetaldehyde and 23 mmol acetaldehyde were incubated with 1.5 kU of wild-type and mutant DERA F2001, respectively, in a total volume of 50 ml containing 0.1 M NaHCO₃ buffer (pH 7.2) at room temperature and with gentle stirring. The reactions were run over five hours and 100 μl samples were drawn at different time points in the course of the reactions. The enzymatic reaction in the samples was stopped after these 5 hours by addition of 900 μl acetonitrile and centrifugation for 10 minutes at 16.000×g. The supernatants were analysed by gas chromatography on a Chrompack CP-SIL8CB column (Varian) using a FID detector for their CTeHP and CHBA content. The respective concentrations determined in these samples can be found in table 6.

The E. coli K12 DERA mutant F2001 exhibits 81 and 86 percent conversion of the present chloroacetaldehyde to CTeHP after two and four hours, respectively, when 150 Upper mmol chloroacetaldehyde are employed. With U is meant one Unit of enzyme, which is the amount of enzyme necessary to convert 1 μmol 2-deoxy-D-ribose 5-phosphate within 1 minute under the conditions of the DERA Natural Substrate Activity Assay. Only in the beginning of the reaction small amounts of the intermediate CHBA are detectable. No CHBA and only small amounts of CTeHP are detectable in the reaction with 150 U of wild-type E. coli K12 DERA per mmol chloroacetaldehyde. For the wild-type DERA seven and eight percent conversion of chloroacetaldehyde to CTeHP are found after two and four hours of incubation time, respectively. Therefore within the same time frame the discovered E. coli K12 mutant DERA F200I showed approximately eleven to twelve fold higher conversions than the wild-type DERA from E. coli K12.

TABLE 6
CTeHP and CHBA formation by E. coli K12 wild-type and mutant
DERA F200I with 150 U per mmol chloroacetaldehyde, respectively.

| | | | CTeHP | CHBA |
|---|---|---|---|---|
| time | CTeHP F200I | CHBA F200I | wild-type | wild-type |
| [h] | [mol/l] | [mol/l] | [mol/l] | [mol/l] |
| 0 | 0.093 | 0.020 | — | — |
| 0.5 | 0.127 | 0.020 | 0.010 | — |
| 1 | 0.148 | 0.011 | 0.013 | — |
| 2 | 0.162 | — | 0.014 | — |
| 4 | 0.172 | — | 0.016 | — |
| 5 | 0.171 | — | 0.015 | — |

(— = below detection limit)

### Example 4

### Saturation Mutagenesis of F200 of Wild-Type E. coli K 2 DERA

### Introduction of F200X Point Mutations

The exchange of the DNA sequence coding for the amino acid residue phenylalanine at position 200 of the E. coli K 2 wild-type DERA amino acid sequence [SEQ ID No. 1] in the E. coli K12 wild-type deoC gene [SEQ ID No. 6] to all possible 64 coding sequences (with X defined as the 20 proteinogenic amino acids as listed above and 3 termination codons) was carried out using the QuikChange Site-Directed Mutagenesis Kit (Stratagene) according to the supplier's manual with the mutagenesis primers

| | |
|---|---|
| F200X_for43 | |
| 5′ GC GTA GAA AAA ACC GTT GGT NNN AAA CCG GCG GGC GGC GTG CG 3′ | [SEQ ID No .9] |
| F200X_rev43 | |
| 5′ CG CAC GCC GCC CGC CGG TTT NNN ACC AAC GGT TTT TTC TAC GC 3′ | [SEQ ID No. 10] |

(with N standing for any of the 4 nucleotides A, C, G and T). As template the E. coli K12 wild-type deoC gene was used, which had been cloned into the NcoI and EcoRI restriction sites of the multiple cloning site of plasmid pBAD/Myc-HisC (Invitrogen) according to the procedure described in WO03/006656.

The resulting PCR products were DpnI digested as described in the supplier's protocol and subsequently used to transform OneShot TOP10 chemically competent E. coli cells (Invitrogen). After plating on selective LB medium containing 100 μg/ml carbenicillin, randomly chosen, independent colonies were used to inoculate 4 deep-well microtiter plates containing 1 ml of 2*TY medium supplemented with 100 μg/ml carbenicillin using one independent colony per well. On each plate three wells were inoculated with E. coli TOP10 colonies harbouring pBAD/Myc-His C with the cloned E. coli wild-type deoC gene [SEQ ID No. 6] and the E. coli deoC gene showing the T706A mutation of [SEQ ID No. 6] resulting in the amino acid exchange of phenylalanine to isoleucine at position 200 of the E. coli DERA amino acid sequence [SEQ ID No. 1], respectively, serving as controls.

### Cultivation, Expression and Screening of the F200X Library

The inoculated deep-well microtiter plates were incubated on a Kühner ISF-1-W gyratory shaker (50 mm shaking amplitude) at 25° C. and 300 rpm for 2 days and used as precultures for the expression cultures of the mutated deoC variants in deep-well microtiter plates. For this purpose 65 μl of each well was transferred into the corresponding well of deep-well microtiter plates containing 935 μl sterile 2*TY medium supplemented with 100 μg/ml carbenicillin and 0.02% (w/v) L-arabinose to induce gene expression.

The expression-cultures were subsequently incubated on a Kühner ISF-1-W gyratory shaker for 24 hours (50 mm shaking amplitude; 37° C.; 300 rpm). Cell harvest and lysis were carried out as described in example 2, except that a total volume of 500 μl lysis buffer was used per well. Substrate incubation was performed as in example 2, but for 20 hours. The reactions were stopped by addition of 1 ml acetonitrile containing 1000 ppm cyclohexylbenzene, which served as internal standard for product quantification in the GC/MS analysis, to each well. Prior to product quantification by GC/MS analysis performed as described in example 2, proteins were precipitated by centrifugation (5,000 rpm at 4° C. for 30 minutes).

In total 14 clones with an at least 2.5 times elevated CTeHP formation were identified (see table 7). Out of these 14 clones 7 contained mutations of F200 for valine, 6 for isoleucine and 1 for methionine, with all possible codons for each of the three amino acids, respectively. According to DNA sequencing results of all these 14 clones, no additional mutations in the deoC genes had occurred.

Retest of F200X “Hits” with the DERA Productivity Factor Test

These 14 clones were retested in comparison to E. coli K12 wild-type DERA according to the DERA Productivity Factor Test as described above. For this purpose the 14 clones were cultivated on 50 ml scale and cell-free extract was prepared as described in Example 2 except that the E. coli TOP10/pBAD/Myc-His C based system was used and expression of the E. coli K12 deoC gene variants was induced by addition of 0.02% (w/v) L-arabinose in the mid-log growth phase instead of by 1 mM IPTG.

The F200V variants showed comparable CTeHP formation in the screening and DERA Productivity Factors as the F2001 variants obtained from this screening. The F200M variant exhibited a slightly lower DERA Productivity Factor than F200V and F200I variants, but which was still more than 10 times increased (more than 1000%) compared to the E. coli K12 wild-type DERA Productivity Factor.

TABLE 7
Screening CTeHP formation and DERA Productivity Factor
of Escherichia coli K12 DERA F200X enzyme mutants
and the E. coli K12 wild-type DERA

| | | | relative CTeHP | DERA |
|---|---|---|---|---|
| | amino acid | | formation [as % | Productivity |
| clone | exchange | codon | wild-type] | Factor |
| Wild-type | none | TTC | 100 | 10 |
| 1-C1 | Val | GTA | 330 | 145 |
| 1-D10 | Met | ATG | 671 | 111 |
| 1-E8 | Val | GTA | 1,041 | 159 |
| 1-E9 | Ile | ATA | 697 | 123 |
| 2-B9 | Val | GTG | 568 | 149 |
| 2-C6 | Ile | ATT | 417 | 145 |
| 2-C11 | Ile | ATA | 428 | 82 |
| 2-E10 | Ile | ATC | 526 | 152 |
| 2-G8 | Val | GTA | 319 | 175 |
| 2-H8 | Val | GTC | 342 | 181 |
| 3-C10 | Ile | ATT | 289 | 163 |
| 3-E5 | Val | GTT | 640 | 154 |
| 4-F6 | Ile | ATA | 250 | 149 |
| 4-H8 | Val | GTG | 382 | 148 |

### Scale-Up of F200X Reactions

To investigate the three of amino acid substitutions F200I, F200V and F200M found by saturation mutagenesis of the F200 position of wild-type E. coli K12 DERA in more detail, defined amounts of cell-free extracts of selected clones were investigated for their performance in CTeHP formation at chloroacetaldehyde concentrations of 0.6 M with acetaldehyde concentrations of 1.2 M.

Clones 1-D10 (F200M), 2-H8 (F200V) and 3-C10 (F2001) were investigated for their expression level by SDS-PAGE analysis of 15 μg protein in their respective cfes. The expression levels of the mutant enzymes proved to be identical to wild-type E. coli K12 DERA. The enzymatic activity in the DERA natural substrate reaction with 2-deoxy-D-ribose 5-phosphate was 29 U/mg for F200M, 38 U/mg for F200V, 36 U/mg for F200I, and 54 U/mg for wild-type DERA of E. coli K12, respectively.

For the CIAA reaction 3 mg of total protein from the respective cell-free extracts were used in a total volume of 1 ml. All reactions were carried out in a 0.1 M NaHCO₃ buffer (pH 7.2) at room temperature and with gentle stirring. For quantification of CTeHP formation 100 μl samples were drawn at different time points in the course of the reactions. The enzymatic reactions in the samples were stopped by addition of 900 μl acetonitrile (containing 1,000 ppm cyclohexylbenzene as internal standard) and centrifugation for 10 minutes at 16,000×g. The supernatants were analysed by gas chromatography on a Chrompack CP-SIL8CB column (Varian) using a FID detector for their CTeHP content. The results of this analysis are shown in table 8.

TABLE 8
Time course of CTeHP formation (in mol/l) from 0.6 M CIAA and
1.2 M acetaldehyde by cell-free extracts containing wild-type
DERA and DERA mutants F200M (clone 1-D10), F200V (clone 2-H8),
and F200I (3-C10) at 3 mg protein per ml reaction volume.

| time [h] | wild-type | F200I | F200V | F200M |
|---|---|---|---|---|
| 0 | — | — | — | — |
| 0.5 | — | 0.14 | 0.15 | 0.09 |
| 1 | — | 0.29 | 0.31 | 0.20 |
| 2 | — | 0.45 | 0.47 | 0.37 |
| 4 | — | 0.49 | 0.49 | 0.45 |
| 5.5 | — | 0.48 | 0.51 | 0.49 |
| 26 | — | 0.51 | 0.52 | 0.45 |

(— = below detection limit)

These results prove that the F200I, the F200V and the F200M substitution are beneficial mutations at amino acid position F200 for the conversion of CIAA and acetaldehyde to CTeHP.

### Example 5

### F2001 Mutation Combined with ΔY259; F2001 Mutation Combined with Δ259 and C-Terminal Extension with [SEQ ID No. 3]

The F2001 exchange was recombined with (i) the deletion of the C-terminal Y259 residue and (ii) its substitution plus extension of the C-terminus of E. coli K12 DERA by the amino acid sequence KTQLSCTKW [SEQ. ID No. 3], respectively, using a PCR based site-directed mutagenesis approach. PCR primers of approximately 30 to 50 nucleotides comprising the respective mutations were synthesized in forward and reverse direction, respectively. In two separate PCR reactions these mutagenesis primers were used on the wild-type deoC gene from E. coli K12 [SEQ ID No. 6] cloned in pDEST14 (Invitrogen) in combination with Gateway system (Invitrogen) specific forward and reverse primer or additional mutagenesis forward and reverse primers, respectively.

| | |
|---|---|
| Gateway system specific forward primer sequence: | |
| 5′ GGG GAC AAG TTT GTA CAA AAA AGC AGG CTT CGA AGG 3′ | [SEQ ID No. 11] |
| Gateway system specific reverse primer sequence: | |
| 5′ GGG GAC CAC TTT GTA CAA GAA AGC TGG GTC 3′ | [SEQ ID No. 12] |
| F200I Forward: | |
| 5′ CCG TTG GTA TCA AAC CGG CGG GCG G 3′ | [SEQ ID No. 13] |
| F200I Reverse: | |
| 5′ CCG CCC GCC GGT TTG ATA CCA ACG G 3′ | [SEQ ID No. 14] |
| ΔY259 Reverse: | |
| 5′ GGG GAC CAC TTT GTA CAA GAA AGC TGG GTC TTA GTA GTG CTG GCG | [SEQ ID No. 15] |
| CTC TTA CC 3′ | |
| C-Extension3 Reverse: | |
| 5′ GGG GAC CAC TTT GTA CAA GAA AGC TGG GTC CTA TTA GTT AGC TGC TGG | [SEQ ID No. 16] |
| CGC TC 3′ | |

The generated partial deoC gene fragments were gel purified, to prevent contamination of subsequent PCR reactions with template deoC fragment DNA. The obtained fragments were used in a PCR reaction to reassemble the variant full-length deoC gene fragments containing the desired mutations. The full-length variant deoC fragments were then subcloned into the pDEST14 vector, according to the supplier's one-tube protocol. The inserts were entirely sequenced to confirm that no unwanted alterations had occurred in the desired E. coli K12 deoC mutant expression constructs.

The obtained E. coli K12 DERA variants F2001/ΔY259 and F2001/ΔY259+SEQ ID No. 3 showed very little catalytic activity towards 2-deoxy-D-ribose 5-phosphate according to the DERA Natural Substrate Activity Assay in the absence of chloroacetaldehyde. Therefore the overexpressed DERA variants were purified by ion-exchange chromatography and ammonium sulphate fractionation according to a procedure as described by Wong and coworkers in J. Am. Chem. Soc. 117 (12), 3333-3339 (1995). The recombined variants F2001+ΔY259 and F2001+ΔY259+SEQ ID No. 3 were compared to DERA variant F2001 and E. coli K12 wild-type DERA for CTeHP synthesis as described in example 3, except that a defined amount of 2.5 mg of the respective purified DERAs (wild-type or variant) was used per ml reaction volume instead of cell-free extracts as described in examples 3 and 4. At substrate concentrations of 0.5 M CIAA and 1.0 M acetaldehyde 61 and 70 percent conversion of the supplied aldehydes to CTeHP were obtained with purified F200I/ΔY259 and F2001/ΔY259+SEQ ID No. 3 after 8 hours, respectively (table 9). With purified F200I a CTeHP concentration of 0.11 M was obtained after 8 hours, corresponding to 23 percent conversion to the desired product. With purified E. coli K12 wild-type DERA very little CTeHP was formed. Here less than seven percent of the supplied aldehydes were converted.

TABLE 9
Comparison of DERA variants F200I, F200I/ΔY259 and F200I/
ΔY259 + SEQ ID No. 3 with E. coli K12 wild-type
DERA for CTeHP formation (in mol/l) with 0.5 M CIAA and 1.0 M
acetaldehyde and 2.5 mg of purified DERAs per ml reaction volume.

| time [h] | wild-type | F200I | F200I/ΔY259 | F200I + SEQ ID No. 3 |
|---|---|---|---|---|
| 0 | 0.011 | 0.003 | 0.021 | 0.029 |
| 0.5 | 0.016 | 0.035 | 0.059 | 0.073 |
| 1 | 0.022 | 0.041 | 0.100 | 0.118 |
| 2 | 0.027 | 0.061 | 0.153 | 0.162 |
| 4 | 0.030 | 0.092 | 0.228 | 0.248 |
| 6 | 0.031 | 0.102 | 0.279 | 0.306 |
| 8 | 0.032 | 0.116 | 0.305 | 0.346 |
| 10 | 0.032 | 0.110 | 0.301 | 0.336 |

### Example 6

### Screening of Wild-Type DERAs for CTeHP Production

Cloning of Wild-Type deoC Genes

The deoC genes coding for the wild-type DERAs of Aeropyrum pernix K1 (GI: 24638457), Bacillus subtilis str. 168 (GI: 1706363), Deinococcus radiodurans R1 (GI: 24636816), and Thermotoga maritima MSB8 (GI: 7674000) were PCR amplified using gene specific primers containing attB recognition sequences for Gateway cloning.

| | |
|---|---|
| A. pernix 5′ forward | |
| 5′ GGG GAC AAG TTT GTA CAA AAA AGC AGG CTT CGA AGG AGA TAG AAC | [SEQ ID No. 17] |
| CAT GAG AGA GGC GTC GGA CGG 3′ | |
| A. pernix 3′ reverse | |
| 5′ GGG GAC CAC TTT GTA CAA GAA AGC TGG GTC TTA GAC TAG GGA TTT GAA | [SEQ ID No. 18] |
| GCT CTC CAA AAC C 3′ | |
| B. subtilis 5′ forward | |
| 5′ GGG GAC AAG TTT GTA CAA AAA AGC AGG CTT CGA AGG AGA TAG AAC | [SEQ ID No. 19] |
| CAT GTC ATT AGC CAA CAT A AT TGA TCA TAC AG 3′ | |
| B. subtilis 3′ reverse | |
| 5′ GGG GAC CAC TTT GTA CAA GAA AGC TGG GTC TTA ATA GTT GTC TCC GCC | [SEQ ID No. 20] |
| TGA TGC 3′ | |
| D. radiodurans 5′ forward | |
| 5′ GGG GAC AAG TTT GTA CAA AAA AGC AGG CTT CGA AGG AGA TAG AAC | [SEQ ID No. 21] |
| CAT GTC ACT CGC CTC CTA CAT CGA CC 3′ | |
| D. radiodurans 3′ reverse | |
| 5′ GGG GAC CAC TTT GTA CAA GAA AGC TGG GTC TCA GTA GCC GGC TCC | [SEQ ID No. 22] |
| GTT TTC GC 3′ | |
| T. maritima 5′ forward | |
| 5′ GGG GAC AAG TTT GTA CAA AAA AGC AGG CTT CGA AGG AGA TAG AAC C | [SEQ ID NO. 23] |
| ATG ATA GAG TAC AGG ATT GAG GAG G 3′ | |
| T. maritima 3′ reverse | |
| 5′ GGG GAC CAC TTT GTA CAA GAA AGC TGG GTC TCA ACC TCC ATA TCT CTC | [SEQ ID NO. 24] |
| TTC TCC 3′ | |

The four wild-type deoC genes were cloned into pDEST14 according to the supplier's protocol and chemically competent E. coli Rosetta (DE3) (Novagen) transformed with the respective pDEST14-deoC constructs. E. coli Rosetta (DE3) strains bearing pDEST14-Ecol-deoC and pDEST14_{—}9-11H, containing the E. coli K12 wild-type deoC gene and the mutated E. coli K12 deoC gene showing the T706A mutation of [SEQ ID No. 6] resulting in the amino acid exchange of phenylalanine to isoleucine at position 200 of the E. coli DERA amino acid sequence [SEQ ID No. 1], respectively, served as controls. Eight randomly chosen, independent colonies of each of these six strains from LB agar plates (containing 100 μg/ml carbenicillin and 35 μg/ml chloramphenicol) were used to inoculate a deep-well microtiter plate containing 1 ml 2*YT medium supplemented with 100 μg/ml carbenicillin and 35 μg/ml chloramphenicol.

### Cultivation, Expression and Screening of Wild-Type DERAs

The inoculated deep-well microtiter plates were incubated on a Kühner ISF-1-W gyratory shaker (50 mm shaking amplitude) at 20° C. and 300 rpm for 2 days and used as precultures for the expression cultures of the mutated deoC variants in deep-well microtiter plates. For this purpose 65 μl of each well was transferred into the corresponding well of deep-well microtiter plates containing 935 μl sterile 2*TY medium supplemented with 100 μg/ml carbenicillin, 35 μg/ml chloramphenicol and 1 mM IPTG to induce gene expression.

The expression-cultures were subsequently incubated on a Kühner ISF-1-W gyratory shaker for 24 hours (50 mm shaking amplitude; 25° C.; 300 rpm). Cell harvest and lysis were carried out as described in example 2, except that a total volume of 500 μl was used and the lysis buffer consisted of 50 mM MOPS buffer pH 7.5 containing 0.1 mg/ml DNAse I (Roche), 2 mg/ml lysozyme (Sigma), 10 mM dithiothreitol (DTT) and 5 mM MgSO₄. Substrate incubation was performed as in example 2, but for 2.5 hours and with substrate concentrations of 0.2 M chloroacetaldehyde and 0.4 M acetaldehyde. The reactions were stopped by addition of 1 ml acetonitrile containing 1000 ppm cyclohexylbenzene, which served as internal standard for product quantification in the GC/MS analysis, to each well. Prior to product quantification by GC/MS analysis performed as described in example 2, proteins were precipitated by centrifugation (5,000 rpm at 4° C. for 30 minutes). Under the employed screening conditions significant DERA activity and CHBA formation could be detected in wells with E. coli K12 wild-type DERA, E. coli K12 DERA variant F2001 and the Bacillus subtilis str. 168 DERA. Under this screening conditions the other wild-type DERAs neither showed activity in the DERA Natural Substrate Assay nor CHBA or CTeHP production in the productivity screening method. The mean value of CHBA formation for E. coli K 2 DERA variant F2001 was about a factor four higher than the CHBA formation by E. coli K12 wild-type DERA and therefore comparable to the values obtained in the same strain background in example 2. Additionally the B. subtilis str.168 wild-type DERA exhibited a 50% higher CHBA production than the wild-type DERA from E. coli K12 with slightly lower DERA Natural Substrate Activity (table 10). This means, that also wild-type DERAs with higher productivity than E. coli K12 DERA having SEQ ID No. 1 and capable of synthesizing CHBA and CTeHP can be found by the GC/MS based productivity method as used and described in example 2.

TABLE 10
Screening of wild-type DERAs for better CHBA formation: DERA
Natural Substrate Activity and relative CHBA formation

| | | Relative CHBA |
|---|---|---|
| | DERA Natural | formation [as |
| | Substrate Assay | % E. coli K12 |
| DERA origin | Activity [U/ml] | wild-type DERA] |
| E. coli K12 wild-type | 4.9 | 100 |
| E. coli K12 F200I | 6.3 | 390 |
| Bacillus subtilis wild-type | 4.2 | 153 |

1. Isolated mutants of enzymes from the group of 2-deoxy-D-ribose 5-phosphate aldolase wild-type enzymes from natural sources belonging to the group consisting of eukaryotic and prokaryotic species, each such wild-type enzyme having a specific productivity factor, as determined by the DERA Productivity Factor Test, in the production of 6-chloro-2,4,6-trideoxy-D-erythrohexapyranoside (CTeHP) from an at least equimolar mixture of acetaldehyde and chloroacetaldehyde, wherein the isolated mutants have a productivity factor which is at least 10% higher than the productivity factor for the corresponding wild-type enzyme from which it is a mutant and wherein the productivity factors of both the mutant and the corresponding wild-type enzyme are measured under identical conditions.

2. Isolated mutants from the group of 2-deoxy-D-ribose 5-phosphate aldolase wild-type enzymes according to claim 1, wherein the isolated mutants have a productivity factor which is at least 10% higher than the productivity factor for the 2-deoxy-D-ribose 5-phosphate aldolase from Escherichia coli K12 (EC4.1.2.4) having the wild type enzyme sequence of [SEQ ID No. 1], and wherein the productivity factors of both the mutant and the Escherichia coli K12 enzyme are measured under identical conditions.

3. Isolated mutants from the group of 2-deoxy-D-ribose 5-phosphate aldolase wild-type enzymes according to claim 1, wherein the mutants are mutants of the 2-deoxy-D-ribose 5-phosphate aldolase from Escherichia coli K12 (EC 4.1.2.4) having the wild-type enzyme sequence of [SEQ ID No. 1].

4. Isolated mutants from the group of 2-deoxy-D-ribose 5-phosphate aldolase wild-type enzymes according to claim 1, wherein the mutants have at least one amino acid substitution at one or more of the positions K13, T19, Y49, N80, D84, A93, E127, A128, K146, K160, I166, A174, M185, K196, F200, or S239 in or at positions corresponding thereto, and/or a deletion of at least one amino acid at one of the positions S258 or Y259 in [SEQ ID No. 1] or at positions corresponding thereto, optionally in combination with C-terminal extension and/or in combination with N-terminal extension.

5. Isolated mutant from the group of 2-deoxy-D-ribose 5-phosphate aldolase wild-type enzymes according to claim 1, wherein the mutants have at least one of the amino acid substitutions in, or corresponding to the substitutions in, [SEQ ID No. 1] selected from the group consisting of:
a. K13 and/or K196 replaced by a positively charged amino acid, preferably by R or H;
b. T19 and/or M185 replaced by another amino acid, preferably by another amino acid selected from the groups consisting of hydrophilic amino acids, in particular consisting of S, T, C, Q, and N, and/or hydrophobic amino acids, in particular consisting of V, L and I;
c. Y49 replaced by an aromatic amino acid selected from the group consisting of F and W;
d. N80 and/or I166 and/or S239 replaced by another amino acid selected from the group of hydrophilic amino acids consisting of T, S, C, Q and N;
e. D84 and/or A93 and/or E127 replaced by another, preferably smaller, amino acid selected from the group of small amino acids consisting of, in order of decreasing size, E, T, N, P, D, C, S, A, and G;
f. A128 and/or K146 and/or K160 and/or A174 and/or F200 replaced by another amino acid selected from the group of hydrophobic amino acids consisting of I, L, M, V, F, and Y;
and/or have a deletion of at least one amino acid at the positions S258 and Y259 in [SEQ ID No. 1], or at positions corresponding thereto, optionally in combination with C-terminal extension and/or in combination with N-terminal extension.

6. Isolated mutant according to claim 4, wherein the C-terminus is extended by one of the fragments TTKTQLSCTKW [SEQ ID No. 2] and KTQLSCTKW [SEQ ID No. 3].

7. Isolated mutant from the group of 2-deoxy-D-ribose 5-phosphate aldolase wild-type enzymes according to claim 5, wherein the mutant has one or more of the mutations in, or corresponding to the mutations in, selected from the group of K13R, T19S, Y49F, N80S, D84G, A93G, E127G, A128V, K146V, K160M, I166T, A174V, M185T, M185V, K196R, F2001, F200M, F200V, S239C, ΔS258, ΔY259, C-terminal extension by TTKTQLSCTKW [SEQ ID No. 2], and C-terminal extension by KTQLSCTKW [SEQ ID No. 3].

8. Isolated mutant from the group of 2-deoxy-D-ribose 5-phosphate aldolase wild-type enzymes according to claim 7, wherein the mutant has at least the following two mutations in, or corresponding to the two mutations in, [SEQ ID No. 1] selected from the group of F2001 and ΔY259; F200M and ΔY259; F200V and ΔY259; F200I and C-terminal extension by KTQLSCTKW [SEQ ID No. 3]; F200M and C-terminal extension by KTQLSCTKW [SEQ ID No. 3]; and F200V and C-terminal extension by KTQLSCTKW [SEQ ID No. 3].

9. Process for the screening for wild-type enzymes from the group of 2-deoxy-D-ribose 5-phosphate aldolase enzymes having a productivity factor, as determined by the DERA Productivity Factor Test, in the production of 6-chloro-2,4,6-trideoxy-D-erythrohexapyranoside (CTeHP) from an at least equimolar mixture of acetaldehyde and chloroacetaldehyde, which is at least 10% higher than the productivity factor for the 2-deoxy-D-ribose 5-phosphate aldolase enzyme from Escherichia coli K12 (EC 4.1.2.4) having a wild-type enzyme sequence of [SEQ ID No. 1], wherein
(A) subsequently (i) total and/or genomic DNA and/or cDNA is isolated; (ii) an expression library of said isolated DNA is prepared, consisting of individual clones comprising said isolated DNA; (iii) the individual clones from the obtained expression library are incubated with a mixture of the substrates acetaldehyde and chloroacetaldehyde; (iv) one or more of the genes from one or more of the clones showing conversion of these substrates into 4-chloro-3-(S)-hydroxy-butyraldehyde (CHBA) and/or 6-chloro-2,4,6-trideoxy-D-erythrohexapyranoside (CTeHP) are isolated and re-cloned into the same genetic background as for [SEQ ID No. 6]; and wherein
(B) the DERA enzymes encoded by the re-cloned genes obtained in step (iv) are expressed and tested by means of the DERA Productivity Factor Test, thereby obtaining a productivity factor for each of such wild-type enzymes; and wherein
(C) the productivity factor for these wild-type enzymes from step (B) is compared to that of the wild-type enzyme from Escherichia coli K12 (EC 4.1.2.4) having a sequence of [SEQ ID No. 1], and one or more genes encoding a DERA enzyme having at least 10% higher productivity factor in the said comparison are selected and isolated.

10. Process for the screening for mutant enzymes from the group of 2-deoxy-D-ribose 5-phosphate aldolase enzymes having a productivity factor, as determined by the DERA Productivity Factor Test, in the production of 6-chloro-2,4,6-trideoxy-D-erythrohexapyranoside (CTeHP) from an at least equimolar mixture of acetaldehyde and chloroacetaldehyde, which is either at least 10% higher than the productivity factor for the corresponding wild-type enzyme or is at least 10% higher than the productivity factor for the 2-deoxy-D-ribose 5-phosphate aldolase enzyme from Escherichia coli K12 (EC 4.1.2.4) having a wild-type enzyme sequence of [SEQ ID No. 1], wherein
(A) subsequently (i) genes encoding a wild-type 2-deoxy-D-ribose 5-phosphate aldolase enzyme are mutated and cloned, in a manner known per se, into the same genetic background as for the gene encoding E. coli K12 DERA having, respectively into the same genetic background as for the corresponding wild-type gene from which it is a mutant, thereby obtaining an expression library of clones from the mutants thus prepared; and wherein
(B) the DERA-enzymes in the clones are expressed and tested by means of the DERA Productivity Factor Test, thereby obtaining a productivity factor for each of the mutant enzymes; and wherein
(C) the productivity factor for the mutant enzymes is compared to that for the corresponding wild-type enzyme, or to that of the wild-type enzyme from Escherichia coli K12 (EC 4.1.2.4) having a sequence of, and one or more genes encoding a DERA mutant having at least 10% higher productivity factor in the respective comparison are selected and isolated.

11. Process according to claim 10, wherein after step (A) (i), in step A (ii) the individual clones from the obtained expression library are incubated with a mixture of the substrates acetaldehyde and chloroacetaldehyde, after which in step A (iii) one or more of the clones showing highest conversion of these substrates into 4-chloro-3-(S)-hydroxy-butyraldehyde (CHBA) and/or 6-chloro-2,4,6-trideoxy-D-erythrohexapyranoside (CTeHP) are selected and wherein the selected clones are used in step B.

12. Isolated nucleic acid obtainable by the screening process of claim 10.

13. An isolated nucleic acid encoding a mutant 2-deoxy-D-ribose 5-phosphate aldolase enzyme according to claim 1.

14. A vector comprising a nucleic acid according to claim 12.

15. A host cell comprising a mutant from the group of 2-deoxy-D-ribose 5-phosphate aldolase wild-type enzymes according to claim 1 or such mutant enzymes, and/or host cells comprising an isolated nucleic acid and/or comprising a vector.

16. Process for the preparation of a mutant 2-deoxy-D-ribose 5-phosphate aldolase having a productivity factor which is at least 10% higher than the productivity factor for the corresponding wild-type enzyme and/or for the 2-deoxy-D-ribose 5-phosphate aldolase enzyme from Escherichia coli (EC 4.1.2.4) having a wild-type enzyme sequence of [SEQ ID No. 1], wherein use is made of a nucleic acid according to claim 12, or of a vector, or of host cells.

17. Process for the preparation of a 2,4-dideoxyhexose or a 2,4,6-trideoxyhexose of formula 1 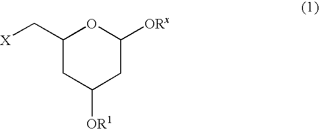
wherein R¹ and R^{x} each independently stand for H or a protecting group and wherein X stands for a halogen; a tosylate group; a mesylate group; an acyloxy group; a phenylacetyloxy group; an alkoxy group or an aryloxy group from acetaldehyde and the corresponding substituted acetaldehyde of formula HC(O)CH₂X, wherein X is as defined above, wherein a mutant DERA enzyme according to claim 1, or a mutant DERA enzyme obtainable by expression of the nucleic acid, or a mutant DERA enzyme, is used and wherein—in case R¹ and/or R^{x} stand for a protecting group, the hydroxy group(s) in the formed compound is/are protected by the protecting group in a manner known per se.

18. Process according to claim 17, wherein the carbonyl concentration, which is the sum of the concentration of aldehyde, 2-substituted aldehyde and the intermediate product formed in the reaction between the aldehyde and the 2-substituted aldehyde (namely a 4-substituted-3-hydroxy-butyraldehyde intermediate), is chosen between 0.1 and 5 moles per liter of reaction mixture.

19. Process according to claim 17, wherein R¹ and R^{x} stand for H.

20. Process for the preparation of a statin using a process according to claim 17 and further process steps known per se.


SEQUENCE LISTING (24 sequences)

SEQ ID NO: 1 - protein, 259 aa, Escherichia coli K12
MTDLKASSLRALKLMDLNTLNDDDTDEKVIALCHQAKTPVGNTAAICIYPRFIPIARKTLKEQGTPEIRIATVTNFPHGNDDIDIALAETRAAIAYGADEVDVVFPYRALMAGNEQVGFDLVKACKEACAAANVLLKVIIETGELKDEALIRKASEISIKAGADFIKTSTGKVAVNATPESARIMMEVIRDMGVEKTVGFKPAGGVRTAEDAQKYLAIADELFGADWADARHYRFGASSLLASLLKALGHGDGKSASSY

SEQ ID NO: 2 - protein, 11 aa, Artificial Sequence
  sequence resulting from synthetic DNA
TTKTQLSCTKW

SEQ ID NO: 3 - protein, 9 aa, Artificial Sequence
  sequence resulting from synthetic DNA
KTQLSCTKW

SEQ ID NO: 4 - DNA, 71 bp, Artificial Sequence
  primer
ggggacaagtttgtacaaaaaagcaggcttcgaaggagatagaaccatgactgatctgaaagcaagcagcc

SEQ ID NO: 5 - DNA, 50 bp, Artificial Sequence
  primer
gggggaccactttgtacaagaaagctgggtcttagtagctgctggcgctc

SEQ ID NO: 6 - DNA, 780 bp, Escherichia coli K12
atgactgatctgaaagcaagcagcctgcgtgcactgaaattgatggacctgaacaccctgaatgacgacgacaccgacgagaaagtgatcgccctgtgtcatcaggccaaaactccggtcggcaataccgccgctatctgtatctatcctcgctttatcccgattgctcgcaaaactctgaaagagcagggcaccccggaaatccgtatcgctacggtaaccaacttcccacacggtaacgacgacatcgacatcgcgctggcagaaacccgtgcggcaatcgcctacggtgctgatgaagttgacgttgtgttcccgtaccgcgcgctgatggcgggtaacgagcaggttggttttgacctggtgaaagcctgtaaagaggcttgcgcggcagcgaatgtactgctgaaagtgatcatcgaaaccggcgaactgaaagacgaagcgctgatccgtaaagcgtctgaaatctccatcaaagcgggtgcggacttcatcaaaacctctaccggtaaagtggctgtgaacgcgacgccggaaagcgcgcgcatcatgatggaagtgatccgtgatatgggcgtagaaaaaaccgttggtttcaaaccggcgggcggcgtgcgtactgcggaagatgcgcagaaatatctcgccattgcagatgaactgttcggtgctgactgggcagatgcgcgtcactaccgctttggcgcttccagcctgctggcaagcctgctgaaagcgctgggtcacggcgacggtaagagcgccagcagctactaa

SEQ ID NO: 7 - DNA, 35 bp, Artificial Sequence
  synthetic DNA
ctactaagacccagctttcttgtacaaagtggtga

SEQ ID NO: 8 - DNA, 30 bp, Artificial Sequence
  synthetic DNA
aagacccagctttcttgtacaaagtggtga

SEQ ID NO: 9 - DNA, 43 bp, Artificial Sequence
  primer
gcgtagaaaaaaccgttggtnnnaaaccggcgggcggcgtgcg

SEQ ID NO: 10 - DNA, 43 bp, Artificial Sequence
  primer
cgcacgccgcccgccggtttnnnaccaacggttttttctacgc

SEQ ID NO: 11 - DNA, 36 bp, Artificial Sequence
  primer
ggggacaagtttgtacaaaaaagcaggcttcgaagg

SEQ ID NO: 12 - DNA, 30 bp, Artificial Sequence
  primer
ggggaccactttgtacaagaaagctgggtc

SEQ ID NO: 13 - DNA, 25 bp, Artificial Sequence
  primer
ccgttggtatcaaaccggcgggcgg

SEQ ID NO: 14 - DNA, 25 bp, Artificial Sequence
  primer
ccgcccgccggtttgataccaacgg

SEQ ID NO: 15 - DNA, 53 bp, Artificial Sequence
  primer
ggggaccactttgtacaagaaagctgggtcttagtagtgctggcgctcttacc

SEQ ID NO: 16 - DNA, 53 bp, Artificial Sequence
  primer
ggggaccactttgtacaagaaagctgggtcctattagttagctgctggcgctc

SEQ ID NO: 17 - DNA, 66 bp, Artificial Sequence
  primer
ggggacaagtttgtacaaaaaagcaggcttcgaaggagatagaaccatgagagaggcgtcggacgg

SEQ ID NO: 18 - DNA, 61 bp, Artificial Sequence
  primer
ggggaccactttgtacaagaaagctgggtcttagactagggatttgaagctctccaaaacc

SEQ ID NO: 19 - DNA, 77 bp, Artificial Sequence
  primer
ggggacaagtttgtacaaaaaagcaggcttcgaaggagatagaaccatgtcattagccaacataattgatcatacag

SEQ ID NO: 20 - DNA, 54 bp, Artificial Sequence
  primer
ggggaccactttgtacaagaaagctgggtcttaatagttgtctccgcctgatgc

SEQ ID NO: 21 - DNA, 71 bp, Artificial Sequence
  primer
ggggacaagtttgtacaaaaaagcaggcttcgaaggagatagaaccatgtcactcgcctcctacatcgacc

SEQ ID NO: 22 - DNA, 53 bp, Artificial Sequence
  primer
ggggaccactttgtacaagaaagctgggtctcagtagccggctccgttttcgc

SEQ ID NO: 23 - DNA, 71 bp, Artificial Sequence
  primer
ggggacaagtttgtacaaaaaagcaggcttcgaaggagatagaaccatgatagagtacaggattgaggagg

SEQ ID NO: 24 - DNA, 54 bp, Artificial Sequence
  primer
ggggaccactttgtacaagaaagctgggtctcaacctccatatctctcttctcc